(19) United States
(12) Patent Application Publication
(10) Pub. No.: US 2025/0220301 A1
(43) Pub. Date: Jul. 3, 2025

(54) TOOTHBRUSH MANUFACTURING CONTROL USING KINETIC IMAGING

(71) Applicant: Colgate-Palmolive Company, New York, NY (US)

(72) Inventors: Xiaonan ZHANG, Hillsborough, NJ (US); Rupert Jack THEOBALD, Basking Ridge, NJ (US)

(73) Assignee: Colgate-Palmolive Company, New York, NY (US)

(21) Appl. No.: 18/852,844

(22) PCT Filed: Mar. 28, 2023
(86) PCT No.: PCT/US2023/016557

§ 371 Date: Sep. 30, 2024

Related U.S. Application Data
(60) Provisional application No. 63/325,228, filed on Mar. 30, 2022.

(51) Int. Cl. H04N 23/661; H04N 23/30

(52) U.S. Cl. CPC H04N 23/661; H04N 23/30

(57) ABSTRACT

Technologies are disclosed for controlling the capture of one or more digital image frames of a product on a conveyor line by a manufacturing control device. The device may be in communication with at least one camera. The device may receive a product position trigger signal. The signal may indicate that the product has entered an imaging zone of the conveyor line. The product may be disposed on the conveyor line in a vertical orientation, a horizontal orientation, and/or an intermediate orientation. The device may determine a shutter trigger sequence for the camera such a predetermined number of the digital image frames are captured while the product is within the imaging zone of the conveyor line. The device may control the capture of the predetermined number of the digital image frames via the camera using the determined shutter trigger sequence upon the receipt of the product position signal.

### CROSS-REFERENCE TO RELATED APPLICATIONS

The present application claims priority to U.S. Provisional Patent Application Ser. No. 63/325,228, filed Mar. 30, 2022, the entirety of which is incorporated herein by reference.

### BACKGROUND

A toothbrush is used to clean the teeth by removing plaque and debris from the tooth surfaces. Conventional toothbrushes having a flat bristle trim are limited in their ability to conform to the curvature of the teeth, to penetrate into the interproximal areas between the teeth, to sweep away the plaque and debris, and to clean along the gum line. Additionally, such toothbrushes have a limited ability to retain dentifrice for cleaning the teeth. During the brushing process, the dentifrice typically slips through the tufts of bristles and away from the contact between the bristles and the teeth. As a result, the dentifrice is often spread around the mouth, rather than being concentrated on the contact of the bristles with the teeth. Therefore, the efficiency of the cleaning process is reduced.

While substantial efforts have been made to modify the cleaning elements of toothbrushes to improve the efficiency of the oral cleaning process, the industry continues to pursue arrangements of cleaning elements that will improve upon the existing technology. In typical oral care implements, bristles having circular transverse cross-sectional profiles are bundled together in a bristle tuft and mounted within tuft holes having circular transverse cross-sectional profiles. However, such a configuration results in gaps being present between adjacent bristles in the tuft and between the bristles of the tuft and the walls of the tuft holes, thereby resulting in a looser packing of the tuft hole and a less than optimal packing factor. These gaps can also reduce the effectiveness of the oral care implement and can cause the oral care implement to effectuate an uncomfortable feeling during brushing. Toothbrush production variations and quality control deficiencies can contribute to lesser-effective oral care implements.

### SUMMARY

Technologies arc disclosed for controlling the capture of one or more digital image frames of a product on a conveyor line, perhaps for example by a manufacturing control device. The manufacturing control device may be in communication with at least one camera.

The device may be configured to receive a product position trigger signal. The signal may indicate that the product has entered an imaging zone of the conveyor line. The product may be disposed on the conveyor line in a vertical orientation, a horizontal orientation, and/or an intermediate orientation (e.g., some orientation between a vertical orientation and a horizontal orientation).

The device may be configured to determine a shutter trigger sequence for the at least one camera. The configuration may cause at least a predetermined number of the digital image frames to be captured while the product is within the imaging zone of the conveyor line.

The device may be configured to control the capture of at least the predetermined number of the digital image frames via the at least one camera using the determined shutter trigger sequence, perhaps for example upon the receipt of the product position signal.

In one or more scenarios, the device may be configured such that the determination of the shutter trigger sequence for the at least one camera may further include a determination an image frame duration for each of the predetermined number of the digital image frames.

In one or more scenarios, the device may be configured such that the determination of the shutter trigger sequence for the at least one camera may be based, at least in part, on at least one image frame rate of the at least one camera.

In one or more scenarios, the camera may comprise a fixed focus lens.

In one or more scenarios, the product may be disposed on the conveyor line in a vertical orientation, a horizontal orientation, and/or an intermediate orientation. The device may be configured to receive a conveyor line speed. In one more scenarios, the device may be configured such that the determination that the shutter trigger sequence for the at least one camera may be based, at least in part, on the conveyor line speed.

In one or more scenarios, the at least one camera may further comprise a liquid lens. The liquid lens may have a focus rise time. The device may be configured to determine a focus trigger sequence for the at least one camera such that the at least predetermined number of the digital image frames may be captured at one or more focus points, perhaps for example while the product is within the imaging zone of the conveyor line. In one or more scenarios, the device may be configured such that the determination of the shutter trigger sequence for the at least one camera may be based, at least in part, on the focus rise time.

In one or more scenarios, the product may be disposed on the conveyor line in a vertical orientation, a horizontal orientation, and/or an intermediate orientation. The conveyor line may be stopped during the capture of the at least predetermined number of the digital image frames via the at least one camera.

In one more scenarios, the product may be a toothbrush, a round hairbrush, and/or a non-round hairbrush, or a like product.

In one or more scenarios, the captured digital image frames may be one or more digital still images, and/or one or more digital image frames of a digital video stream, for example.

In one or more scenarios, the device may be configured such that the determined the shutter trigger sequence may control the at least one camera such that at least some of the captured digital image frames may (e.g., each) represent a (e.g., respectively) different focused depth profile of the product, and/or a (e.g., respectively) different focused region of the product.

A manufacturing control system may be configured to control a capture of one or more digital image frames of a product on a conveyor line. The system may comprise a camera. The system may comprise a position detector. The system may comprise a control device. The control device may comprise a memory and/or a processor. The processor may be configured at least to receive a product position trigger signal from the position detector. The signal may indicate that the product has entered an imaging zone of the conveyor line. The processor may be configured to determine a shutter trigger sequence for the camera such that at least a predetermined number of the digital image frames are captured while the product is within the imaging zone of the conveyor line. The processor may be configured to control the capture of at least the predetermined number of the digital image frames via the camera using the determined shutter trigger sequence, perhaps for example upon receipt of the product position signal.

A method to control a capture of one or more digital image frames of a product on a conveyor line may be performed by a manufacturing control system. The method may comprise detecting, by a position detector, that the product has entered an imaging zone of the conveyor line. The method may comprise sending, by the position detector, a product position trigger signal indicating that the product has entered the imaging zone of the conveyor line. The method may comprise receiving, by a control device, the product position trigger signal from the position detector. The method may comprise determining, by the control device, a shutter trigger sequence for the camera such that at least a predetermined number of the digital image frames are captured, perhaps while the product is within the imaging zone of the conveyor line. The method may comprise controlling, by the control device, the capture of at least the predetermined number of the digital image frames via the camera using the determined shutter trigger sequence upon receipt of the product position signal. The method may comprise capturing, by a camera, at least the predetermined number of the digital image frames per the determined shutter trigger sequence.

### BRIEF DESCRIPTION OF DRAWINGS

The elements and other features, advantages and disclosures contained herein, and the manner of attaining them, will become apparent and the present disclosure will be better understood by reference to the following description of various examples of the present disclosure taken in conjunction with the accompanying drawings, wherein:

FIG. 1 is a block diagram illustrating an example manufacturing environment control network operable to control one or more parts of a product manufacturing process via one or more devices, such as a manufacturing control device (MCD) device, among other devices.

FIG. 2A is a collection of examples of product toothbrush head images that illustrate various manufacturing quality control inspection challenges.

FIG. 2B is a collection of examples of product toothbrush head images that illustrate various manufacturing quality control inspection challenges.

FIG. 3 is an example flow diagram of at least one technique for capturing one or more image frames of a product in a manufacturing environment.

FIG. 4 is a block diagram of a hardware configuration of an example device that may control one or more parts of a product manufacturing process, such as the MCD device of FIG. 1.

FIG. 5 is an example diagram of a quality control inspection imaging technique using an X-ray camera.

FIG. 6 illustrates an example of a kinetic imaging technique for a product during a manufacturing process.

FIG. 7 illustrates an example of a kinetic imaging technique arrangement for a product during a manufacturing process.

FIG. 8 illustrates an example of a kinetic imaging technique arrangement for a product during a manufacturing process.

FIG. 9 illustrates an example of a kinetic imaging technique arrangement for a product during a manufacturing process.

FIG. 10 is an example block diagram of a kinetic imaging technique integrated into an extended manufacturing quality control scheme.

FIG. 11 is an example block diagram of a kinetic imaging technique integrated into an extended manufacturing quality control scheme.

FIG. 12 is an example illustration of a camera device timing/parameter sequence/pattern for the capture of one or more digital image frames of a product during a manufacturing process.

FIG. 13 is an example illustration of a camera device timing/parameter sequence/pattern for the capture of one or more digital image frames of a product during a manufacturing process.

FIG. 14 is an example block diagram of a kinetic imaging technique integrated into an extended manufacturing quality control scheme.

FIG. 15 is an example block diagram of a kinetic imaging technique integrated into an extended manufacturing quality control scheme.

FIG. 16 is an example illustration of a controlled capture of one or more digital image frames of a product during a manufacturing process.

FIG. 17 illustrates an example composite image that may be constructed from one or more digital images of a product captured during a manufacturing process.

FIG. 18 illustrates an example a camera device liquid lens control configuration and control technique.

FIG. 19 is an example diagram of at least one technique for digital image frame capture light control.

### DETAILED DESCRIPTION

For the purposes of promoting an understanding of the principles of the present disclosure, reference will now be made to the examples illustrated in the drawings, and specific language will be used to describe the same. It will nevertheless be understood that no limitation of the scope of this disclosure is thereby intended.

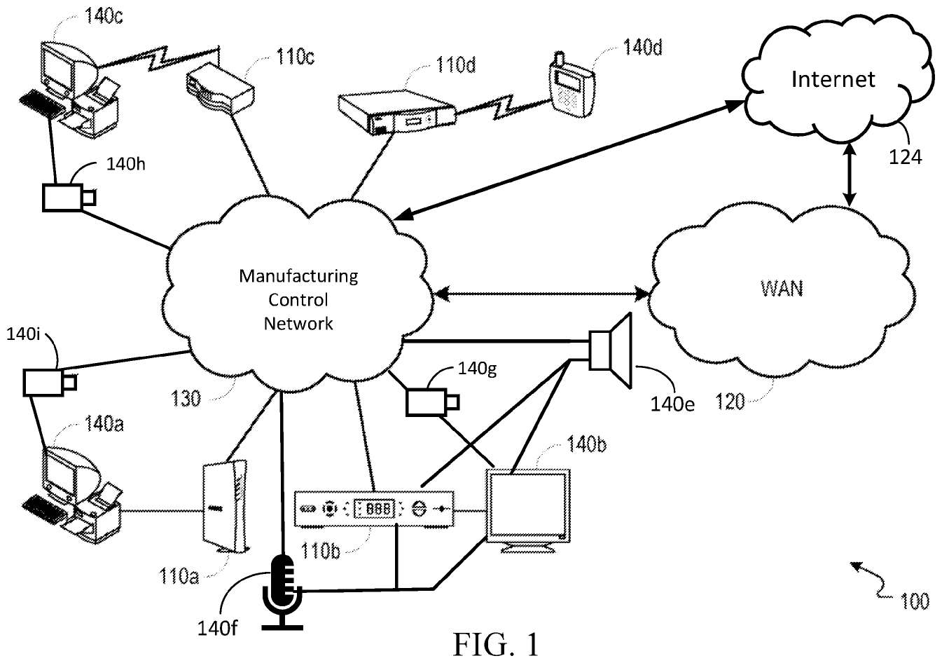

FIG. 1 is a block diagram illustrating an example Manufacturing Environment Control Network 100 operable to monitor and/or control one or more parts of a product manufacturing process. One or more of digital and/or analog control signals, electronic content, various input signals, and or various output signals, among other manufacturing control information may be communicated from/across/among the Manufacturing Environment Control Network 100. One or more of discrete control and/or continuous control schemes, techniques, and/or algorithms may be processed/performed by/across/from the Manufacturing Environment Control Network 100.

Electronic content may include media content, electronic documents, device-to-device communications, streaming media content, digital image still frames, digital streaming video, Internet/cloud-based electronic applications/services/databases, electronic communications/services (e.g., video/audio conferencing), Internet-based electronic services, virtual reality content and/or services, augmented reality content and/or services, media captioning content and/or services, electronic commerce, video components/elements of electronic content, and/or audio components/elements of electronic content, among other types of electronic content.

In one or more scenarios, MCD devices 110a-d transmit/receive signals and/or communications and/or may receive data service(s) from a wide area network (WAN) 120 via a connection to a Manufacturing Control Network 130. The one or more nodes of Manufacturing Control Network 130 and/or the WAN 120 may communicate with one or more cloud-based nodes (not shown) via the Internet 124.

The MCD devices can include, for example, a modem 110a, a process control device/logic controller 110b, a wireless router including an embedded modem 110c, or a media gateway 110d, among many others (e.g., digital subscriber line (DSL) modem, voice over internet protocol (VOIP) terminal adapter, video game console, digital versatile disc (DVD) player, communications device, hotspot device, etc.). The Manufacturing Control Network 130, for example, can be a hybrid fiber-coaxial (HFC) network, a local area network (LAN), a wireless local area network (WLAN), a cellular network, a personal area network (PAN), as well as others. As used herein, a manufacturing control device may any of the devices 110a-110d and/or 140a-140i, an Internet Gateway, a router device, a set-top box (STB), a process control device/logic controller, a smart media device (SMD), a cloud computing device, any type of MCD, and/or any other suitable device (e.g., wired and/or wireless) that may be configured to perform one or more of the techniques and/or functionality disclosed herein, for example.

The MCD devices can facilitate communications between the WAN 120 and client devices 140a-140i. A cable modem or embedded MTA (cMTA) 110 a can facilitate communications between the WAN 120 and a computer 140a. A process control device/logic controller 110b can facilitate communications between the WAN 120 and a television/monitor 140b (e.g., a media presentation device, a graphical user interface, a process control interface, etc.) and/or a digital video recorder (DVR). A wireless router 110c can facilitate communications between a computer 140c and the WAN 120.

The media gateway 110d can facilitate communications between a mobile device 140d (e.g., a tablet computing device, a smartphone, a personal digital assistant (PDA) device, a laptop computing device, etc.; one or more devices being PC-based, iOS-based, Linux-based, and/or Android-based, etc.) and the WAN 120. One or more speaker devices (e.g., sound radiation devices/systems) 140e may be in communication with the Manufacturing Control Network 130, process control device/logic controller 110b, and/or television 140b, etc. Camera devices 140g , 140h, and/or 140i may be in communication with the computer 140a, the television 140b, the computer 140c, and/or the Manufacturing Control Network 130, for example, among other devices and networks.

The one or more speaker devices 140e (e.g., surround sound speakers, home theater speakers, other external wired/wireless speakers, loudspeakers, full-range drivers, subwoofers, woofers, mid-range drivers, tweeters, coaxial drivers, etc.) may broadcast at least an audio component of electronic content/media content, among other audio signals, processes, and/or applications. The one or more speaker devices 140e may possess the capability to radiate sound in pre-configured acoustical/physical patterns (e.g., a cone pattern, a directional pattern, etc.). For example, process control device/logic controller manufacturing process audible alarms may be communicated via one or more of the speaker devices 140e.

One or more microphone devices 140f may be external/standalone microphone devices. The one or more microphone devices 140f may be in communication with the Manufacturing Control Network 130, process control device/logic controller 110b, television 140b, computer 140a, computer 140c, mobile device 140a, etc. Any of the client devices 110a-110d and/or devices 140a-140i may include internal microphone devices. The one or more speaker devices 140e (e.g., “speakers”) and/or the one or more microphone devices 140f (e.g., “microphones”, that may be “high quality” devices such as far field microphones, noise-cancelling microphones, shotgun microphones, dynamic microphones, ribbon microphones, and/or various size diaphragm microphones, Bluetooth™-based remote/control devices, RF4CE-based remote/control devices, etc.) may have wired and/or wireless connections (e.g., Bluetooth, Wi-Fi, private protocol communication network, etc.) to any of the other devices 140a-140i, the Manufacturing Control Network 130, the WAN 120, and/or the Internet 124.

The camera devices 140g-140i may provide digital video input/output capability for one or more of the devices 110a-110d and/or devices 140a-140d. The camera devices 140g-140i may communicate with any of the devices 110a-110d and/or devices 140a-140f, perhaps for example via a wired and/or wireless connection. One or more of the camera devices 140g-140i may capture digital images and/or may scan images of various kinds, such as Universal Product Code (UPC) codes and/or Quick Response (QR) codes, for example, among other images. One or more of the camera devices 140g-140i may provide for video input/output for video conferencing (e.g., may serve as webcams or the like), for example, among other video functions.

Any of the camera devices 140g-140i may include microphone devices and/or speaker devices. The input/output of any of the camera devices 140g-140i may include audio signals/packets/components, perhaps for example separate/separable from, or in some (e.g., separable) combination with, the video signals/packets/components of any of the camera devices 140g-140i.

One or more of the camera devices 140g-140i may detect the presence of one or more people and/or things that may be proximate to the camera devices 140g-140i and/or that may be in the same general space (e.g., the same room, same manufacturing space, etc.) as the camera devices 140g-140i. One or more of the camera devices 140g-140i may gauge a general activity level (e.g., high activity, medium activity, and/or low activity) of one or more people that may be detected by the camera devices 140g-140i. One or more of the camera devices 140g-140i may detect one or more general characteristics (e.g., height, body shape, skin color, pulse, heart rate, breathing count, product size, product volume, product bulk, etc.) of the one or more people detected by the camera devices 140g-140i. One or more of the camera devices 140g-140i may be configured to recognize one or more specific people, for example. One or more of the camera devices 140g-140i may be configured to detect a user's/viewer's attention/gaze toward/on a monitor/television device (e.g., detecting a location on the monitor/television device that may correspond to a user's/viewer's attention/gaze toward/on the monitor/television device).

One or more of the camera devices 140g-140i may be use wireless communication with any of the devices 110a-110d and/or 140a-140d, such as for example Bluetooth™ and/or Wi-Fi™, among other wireless communication protocols. One or more of the camera devices 140g-140imay be external to any of the devices 110a-110d and/or devices 140a-140d. One or more of the camera devices 140g-140i may be internal to any of the devices 110a-110d and/or devices 140a-140d.

One or more of the camera devices 140g-140i may be an “X-ray” camera device. The X-ray camera device may be capture high-definition X-ray images (e.g., digital/analog still frames, video, etc.) of objects/products in a manufacturing environment, for example on a conveyor line. The X-ray camera device may capture images of products moving at a relatively high speed, with substantially the same high definition clarity as that of a product moving relatively slowly or not moving at all. For example, the X-ray camera (e.g., manufactured by Hamamatsu such as a C9878 X-ray high speed camera system, or a like/equivalent device) may capture product X-ray images from zero to five hundred meters/minute. The X-ray camera may have one or more parameters configurable remotely and/or locally.

One or more of the camera devices 140g-140i may be an industrial vision camera device. The vision camera may be a gigabit Ethernet compatible device (e.g., 10 GB Ethernet, or the like). The vision camera may function in black & white and/or color. The vision camera may have a capacity of at least 8.8 megapixel, or the like. The vision camera may have a resolution of 4096×2160 pixel, or the like. For example, the vision camera may be a (e.g., manufactured by Baumer such as a VLXT-90C.ILX series, or like/equivalent device) may capture product images in various forms such as digital still image frames and/or video streams, etc., perhaps for example from zero to ninety-five (95) frames per second (fps). The vision camera may have one or more parameters configurable remotely and/or locally.

MCD devices such as process control device/logic controller devices, media gateway devices, among others, may support visual and/or voice interface with users, viewers, and/or process operators. This interface may support smart enhancement to the user/viewer/operator experience, for example in the manufacturing environment, or in any user network environment. One or more traditional and/or current viewer experiences can be enriched to utilize visual and/or voice interface, perhaps for example to derive smart actions and/or results.

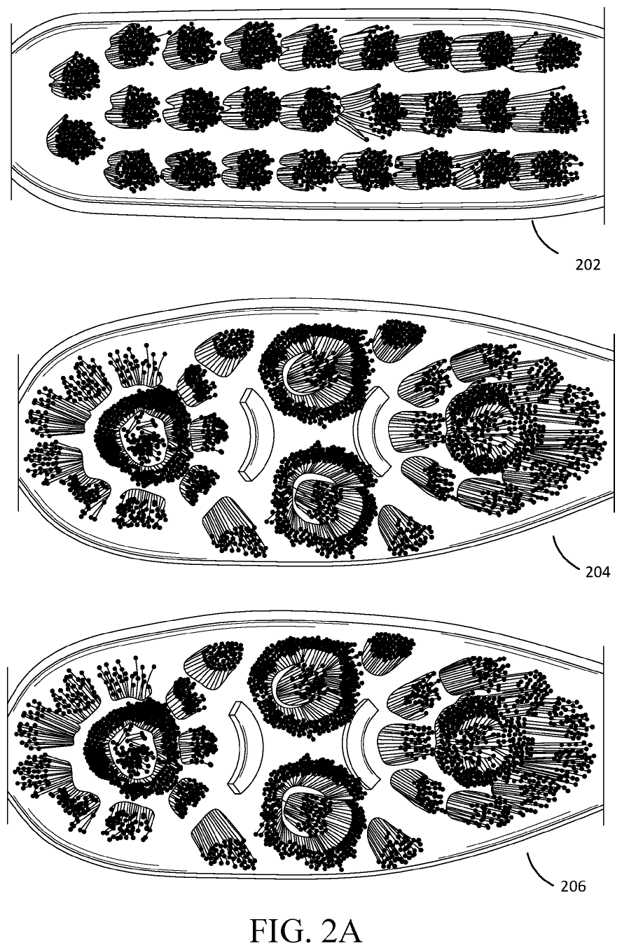

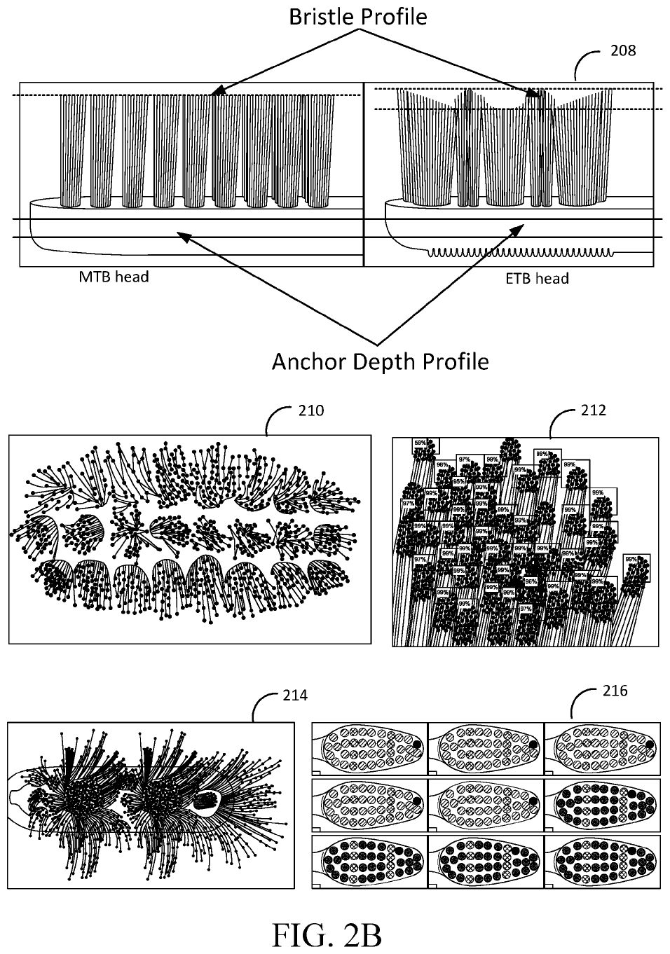

In one or more scenarios, the product under manufacture may be a toothbrush, a hairbrush, a round hairbrush, and/or a non-round hairbrush, or the like. Manufacturing of such brushes, for example, toothbrushes, presents one or more manufacturing quality control challenges. For example, referring to FIG. 2A and FIG. 2B, toothbrush manufacturing includes quality control activities such as anchor filament inspection, tufting variation/defect inspection, end rounding inspection, bristle capacity inspection, filament length inspection, filament top defect inspection, other anchor imperfection inspections, loss tufts inspection, and/or bristle profiling inspection, among other quality control inspections.

In FIG. 2A, toothbrush head image 202 illustrates the challenges to “hands off/hands free” aspects in various quality control inspections, for example for anchor and/or filament inspection. Toothbrush head images 204 and 206 illustrate the challenges to “hands off/hands free” aspects in various quality control inspections, for example for filament length variations. Toothbrush head images 204 and 206 also illustrate the challenges in quality control inspections such as where all the filaments are not in focus throughout the entirety of a particular image of a toothbrush head.

In FIG. 2B, toothbrush head image 208 illustrates the challenges to “hands off/hands free” aspects in various quality control inspections, for example for anchor depth profile inspection and/or bristle profile inspection. Toothbrush head images 210, 212, 214, and 216 illustrate the challenges to “hands off/hands free” aspects in various quality control inspections, for example for filament length variations, bristle profile/composition variations, tufting variations, end rounding variations, and/or bristle pattern variations, among other variations, for example.

Using current methods and/or devices, users/operators and/or product manufacturers might not have the techniques, functions, and/or capability to perform product manufacturing quality control inspections “hands free”, (e.g., relatively) real time, “automatic”, and/or “high-speed”, or the like. Quality control inspections that require “hands on” or “manual” activities may consume large amounts of time that may interfere/slow the manufacturing process and/or may fail to detect product quality issues that could result in distribution of impaired quality products to the consumer, perhaps injuring the product reputation with the consumer, for example.

Technologies that may provide users/operators and/or product manufacturers with an ability and/or function to perform “hands off”, (e.g., relatively) “real time”, “automatic”, and/or “high-speed” product quality control inspections of a product(s) based on one or more images of the product(s) captured during the manufacturing process could be useful.

In one or more scenarios, any of the devices 110a-110d, 140a-140i, among other devices, may be used to implement any of the capabilities, techniques, methods, and/or devices described herein.

The WAN network 120 and/or the Manufacturing Control Network 130 may be implemented as any type of wired and/or wireless network, including a local area network (LAN), a wide area network (WAN), a global network (the Internet), etc. Accordingly, the WAN network 120 and/or the Manufacturing Control Network 130 may include one or more communicatively coupled network computing devices (not shown) for facilitating the flow and/or processing of network communication traffic via a series of wired and/or wireless interconnects. Such network computing devices may include, but are not limited, to one or more access points, routers, switches, servers, computing devices, and/or storage devices, etc.

Without the capabilities, techniques, methods, and/or devices described herein, the skilled artisan would not appreciate how to automatically and/or in real time conduct/obtain “no touch” /“hands free” quality control inspections/inspection results for a product (e.g., a toothbrush) during a high-speed manufacturing process.

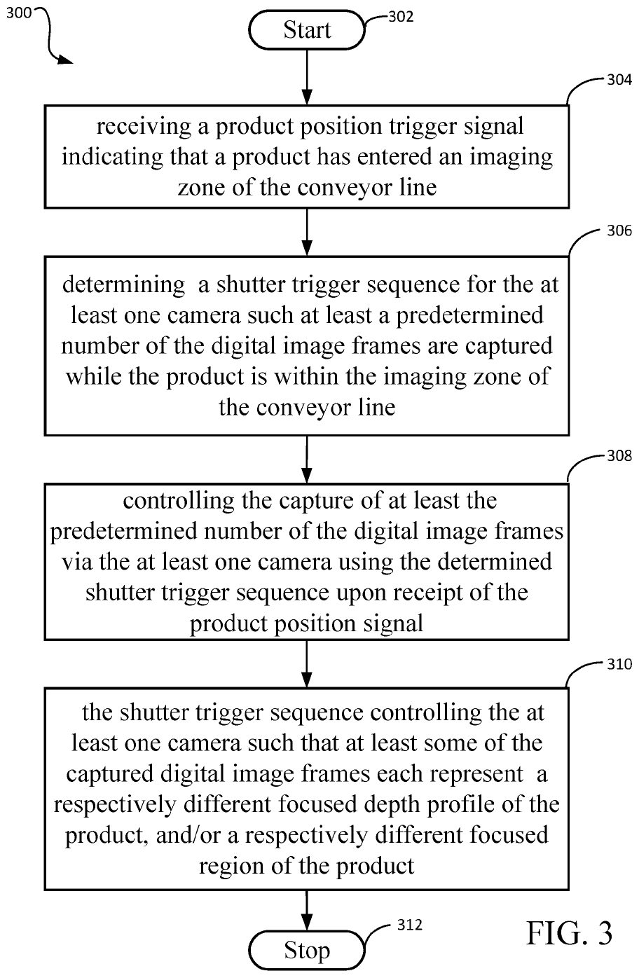

Referring now to FIG. 3, a diagram 300 illustrates an example technique for controlling the capture of one or more digital image frames of a product on a conveyor line. The method may be performed by a manufacturing control device, among other devices. For example, the manufacturing control device may be a process control device/logic controller 110b, among other devices 110a-110d and/or 140a-140i, and/or a cloud computing device. The manufacturing control device may be in communication with at least one camera device and/or a display device. At 302, the process may start or restart.

At 304, the manufacturing control device may receive a product position trigger signal indicating that a product (e.g., a toothbrush) has entered an imaging zone of the conveyor line. At 306, the manufacturing control device may determine a shutter trigger sequence for the at least one camera such at least a predetermined number of the digital image frames may be captured, perhaps for example while the product is within the imaging zone of the conveyor line. The product may be disposed on the conveyor line in a vertical orientation and/or a horizontal orientation, and/or perhaps a position between the vertical orientation and horizontal orientation.

At 308 the manufacturing control device may control the capture of at least the predetermined number of the digital image frames via the at least one camera using the determined shutter trigger sequence, perhaps for example upon receipt of the product position signal.

At 310, the manufacturing control device may control the at least one camera using the determined shutter trigger sequence such that at least some of the captured digital image frames may each represent a respectively different focused depth profile of the product, and/or a respectively different focused region of the product. At 312 the process may stop or restart.

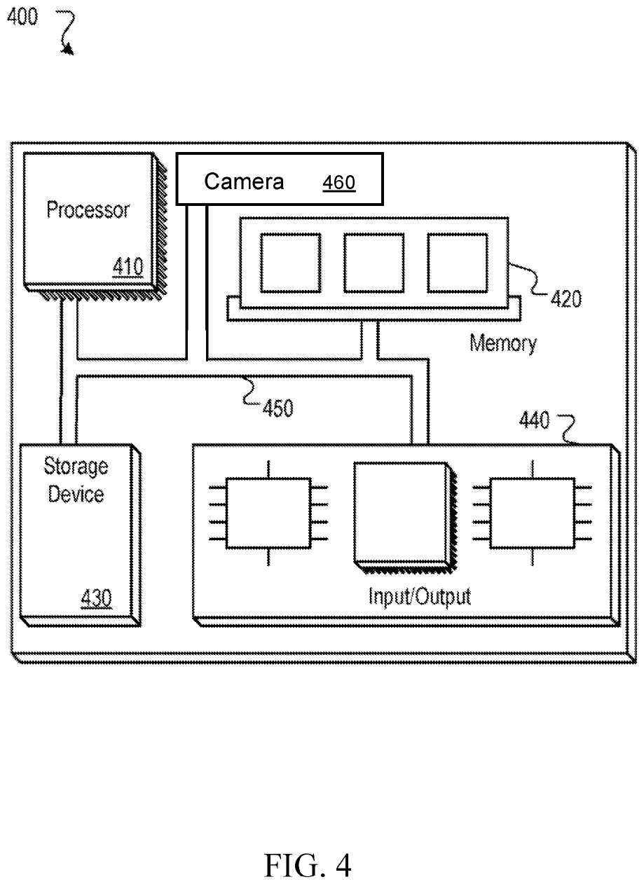

FIG. 4 is a block diagram of a hardware configuration of an example device that may function as a process control device/logic controller, such as the MCD device 110b of FIG. 1, among other devices such as 140a-140i, and any of the devices 110a-110d, for example. The hardware configuration 400 may be operable to facilitate delivery of information from an internal server of a device. The hardware configuration 400 can include a processor 410, a memory 420, a storage device 430, and/or an input/output device 440. One or more of the components 410, 420, 430, and 440 can, for example, be interconnected using a system bus 450. The processor 410 can process instructions for execution within the hardware configuration 400. The processor 410 can be a single-threaded processor or the processor 410 can be a multi-threaded processor. The processor 410 can be capable of processing instructions stored in the memory 420 and/or on the storage device 430.

The memory 420 can store information within the hardware configuration 400. The memory 420 can be a computer-readable medium (CRM), for example, a non-transitory CRM. The memory 420 can be a volatile memory unit, and/or can be a non-volatile memory unit.

The storage device 430 can be capable of providing mass storage for the hardware configuration 400. The storage device 430 can be a computer-readable medium (CRM), for example, a non-transitory CRM. The storage device 430 can, for example, include a hard disk device, an optical disk device, flash memory and/or some other large capacity storage device. The storage device 430 can be a device external to the hardware configuration 400.

The input/output device 440 may provide input/output operations for the hardware configuration 400. The input/output device 440 (e.g., a transceiver device) can include one or more of a network interface device (e.g., an Ethernet card), a serial communication device (e.g., an RS-232 port), one or more universal serial bus (USB) interfaces (e.g., a USB 2.0 port) and/or a wireless interface device (e.g., an 802.11 card). The input/output device can include driver devices configured to send communications to, and/or receive communications from one or more networks (e.g., Manufacturing Control Network 130 of FIG. 1). The input/output device 400 may be in communication with one or more input/output modules (not shown) that may be proximate to the hardware configuration 400 and/or may be remote from the hardware configuration 400. The one or more output modules may provide input/output functionality in the digital signal form, discrete signal form, TTL form, analog signal form, serial communication protocol, fieldbus protocol communication and/or other open or proprietary communication protocol, and/or the like.

The camera device 460 may provide digital video input/output capability for the hardware configuration 400. The camera device 460 may communicate with any of the elements of the hardware configuration 400, perhaps for example via system bus 450. The camera device 460 may capture digital images and/or may scan images of various kinds, such as Universal Product Code (UPC) codes and/or Quick Response (QR) codes, for example, among other images as described herein. In one or more scenarios, the camera device 460 may be the same and/or substantially similar to any of the other camera devices described herein.

The camera device 460 may include at least one microphone device and/or at least one speaker device. The input/output of the camera device 460 may include audio signals/packets/components, perhaps for example separate/separable from, or in some (e.g., separable) combination with, the video signals/packets/components the camera device 460.

The camera device 460 may also detect the presence of one or more people that may be proximate to the camera device 460 and/or may be in the same general space (e.g., the same room) as the camera device 460. The camera device 460 may gauge a general activity level (e.g., high activity, medium activity, and/or low activity) of one or more people that may be detected by the camera device 460. The camera device 460 may detect one or more general characteristics (e.g., height, body shape, skin color, pulse, heart rate, breathing count, etc.) of the one or more people detected by the camera device 460. The camera device 460 may be configured to recognize one or more specific people, for example.

The camera device 460 may be in wired and/or wireless communication with the hardware configuration 400. In one or more scenarios, the camera device 460 may be external to the hardware configuration 400. In one or more scenarios, the camera device 460 may be internal to the hardware configuration 400.

In one or more scenarios, one or more techniques may provide for control of the capture of one or more digital image frames of a product (e.g., a toothbrush) on a conveyor line, for example. One or more techniques may be performed by a manufacturing control device, among other devices. The manufacturing control device may be in communication with at least one camera.

The manufacturing control device may be configured to receive a product position trigger signal. The product position trigger signal may indicate that the product has entered an imaging zone of the conveyor line. The product could be disposed on the conveyor line in a vertical orientation, a horizontal orientation, and/or an intermediate orientation between the vertical orientation and the horizontal orientation.

The manufacturing control device may be configured to determine a shutter trigger sequence for the at least one camera such at least a predetermined number of the digital image frames may be captured, perhaps for example while the product is within the imaging zone of the conveyor line.

The manufacturing control device may be configured to control the capture of at least the predetermined number of the digital image frames via the at least one camera using the determined shutter trigger sequence, perhaps for example upon receipt of the product position signal.

In one or more scenarios, the manufacturing control device may be configured to determine an image frame duration, perhaps for example for one or more, or each, of the predetermined number of the digital image frames.

In one or more scenarios, the manufacturing control device may be configured such that the shutter trigger sequence for the at least one camera may be based, at least in part, on at least one image frame rate of the at least one camera, for example.

In one or more scenarios, the at least one camera may comprise a fixed focus lens.

In one or more scenarios, perhaps for example where the product may be disposed on the conveyor line in a vertical orientation, a horizontal orientation, and/or an intermediate orientation, among other scenarios, the manufacturing control device may be configured to receive a conveyor line speed (e.g., via the Manufacturing Control Network 130, etc.). The shutter trigger sequence for the at least one camera may be based, at least in part, on the conveyor line speed.

In one or more scenarios, the at least one camera may comprise a liquid lens. The liquid lens may have a focus rise time.

The manufacturing control device may be configured to determine a focus trigger sequence for the at least one camera such that the at least predetermined number of the digital image frames may be captured at one or more focus points, perhaps for example while the product may be within the imaging zone of the conveyor line. The shutter trigger sequence for the at least one camera may be based, at least in part, on the focus rise time.

In one or more scenarios, the product may be disposed on the conveyor line in a vertical orientation, a horizontal orientation, and/or an intermediate orientation, and/or the conveyor line may be stopped during the capture of the at least predetermined number of the digital image frames via the at least one camera.

In one or more scenarios, the product may be a toothbrush, a hairbrush, a non-round hairbrush, and/or the like.

In one or more scenarios, the captured digital image frames may be one or more digital still images, and/or one or more digital image frames of a digital video stream.

In one or more scenarios, the manufacturing control device may be configured such that the determined the shutter trigger sequence may control the at least one camera such that at least some of the captured digital image frames may (e.g., each) represent a (e.g., respectively) different focused depth profile of the product, and/or a (e.g., respectively) different focused region of the product.

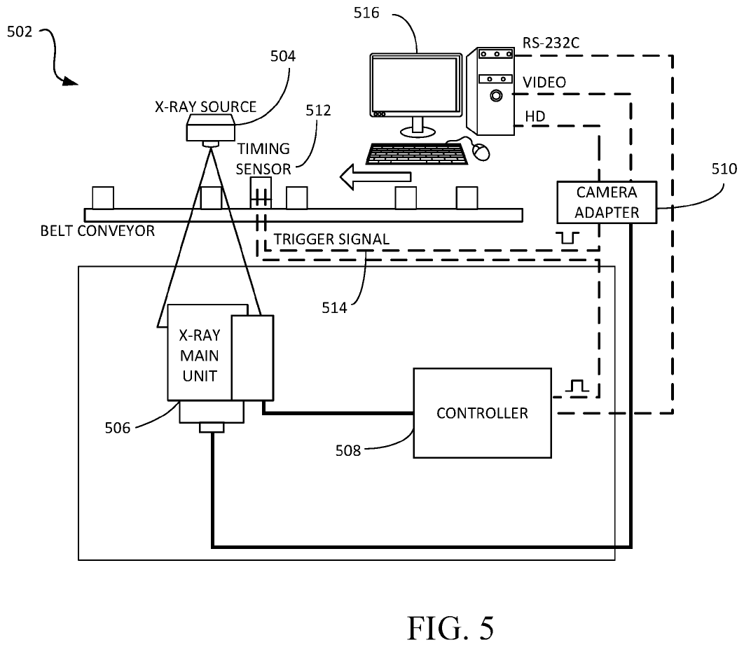

FIG. 5 is an example diagram of a quality control inspection imaging technique using an X-ray camera. Referring to FIG. 5, an example product manufacturing X-ray imaging arrangement 502 may capture one or more X-ray images of a product on a conveyor line, perhaps for example during the product manufacturing process. In one or more scenarios, the X-Ray camera 504 may be at least one of the camera devices 140g-140i, for example. The X-Ray camera 504 may communicate with X-Ray main unit 506 (e.g., by Hamamatsu, or the like, for example). The controller 508 may be Manufacturing Control Device 110b, among other devices, for example. A camera adapter 510 may interface a manufacturing control system node 516 with the controller 508. A timing sensor/product position detector 512 may provide a timing signal 514 to the controller 508 to synchronize the capture of the X-Ray image of the product on the conveyor line.

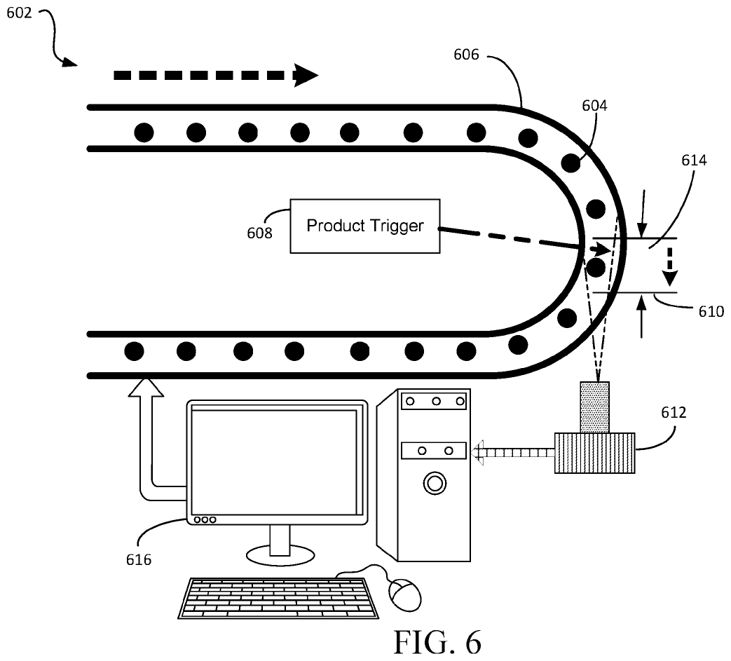

FIG. 6 illustrates an example of a kinetic imaging technique for a product during a manufacturing process. Referring to FIG. 6, an example product manufacturing digital imaging arrangement 602 may capture one or more digital image frames of a product 604 (e.g., a toothbrush) on a conveyor line 606, perhaps for example during the product 604 manufacturing process. Perhaps for example when a product position sensor/trigger 608 detects the product 604 entering an imaging area/zone 610, a (e.g., vision) camera device 612 (e.g., by Olympus, by Bauer Full HD 95FPS, or the like) may be controlled by a process control device/logic controller (not shown) to capture one or more digital image frames of the product 604. For example, at 614 when the product moves into the imaging area 610, the vision camera device 612 may be controlled to capture one or more (e.g., ten or more) digital image frames that may be (e.g., each) focused in on one or more different part/depth/region of the product (e.g., toothbrush).

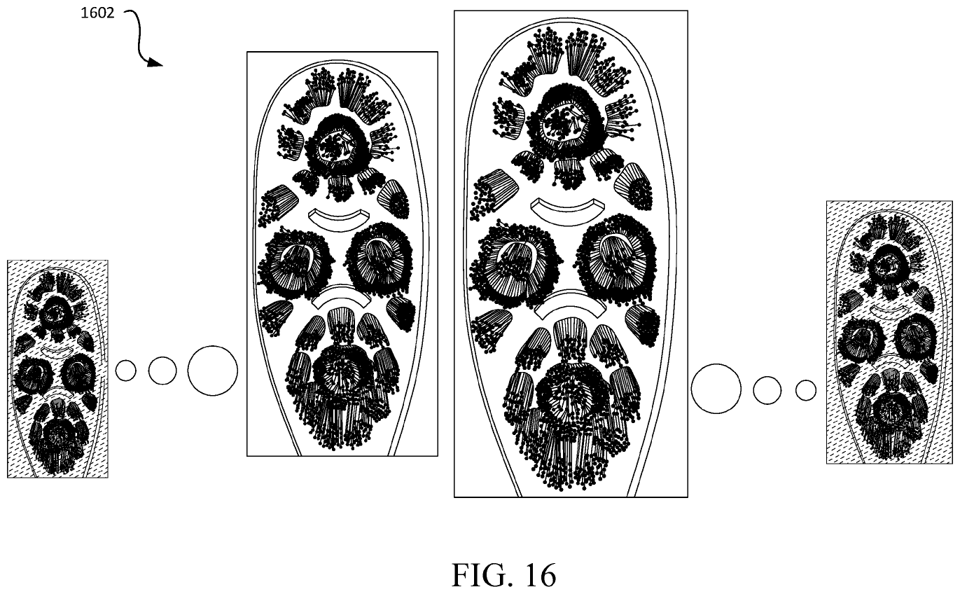

For example, in toothbrush manufacturing scenarios, FIG. 16 illustrates the one or more digital image frames that may be captured by the camera device 612, where at least some of the captured digital image frames may (e.g., each) represent a (e.g., respectively) different focused depth profile of the toothbrush, and/or a (e.g., respectively) different focused region of the toothbrush.

In one more scenarios, the imaging area 610 of the conveyor 606 may be located on a curved area. The product 604 may be carried on the conveyor 606 in a vertical orientation, a horizontal orientation, and/or an intermediate orientation. In the toothbrush manufacturing scenario, the bristle tip of the toothbrush 604 may be arranged to face the camera device 612.

In one or more scenarios, the camera device 612 may comprise a fixed focus lens. The camera device 612 may be controlled to use a shutter trigger sequence to capture the one or more digital image frames of the toothbrush 604 as it moves through/remains within the imaging area 610. The conveyor 606 movement may be used as a kinetic image focus mechanism for the digital image frame capturing technique. In one or more scenarios, the camera device 612 may be at least one of the camera devices 140g-140i, for example.

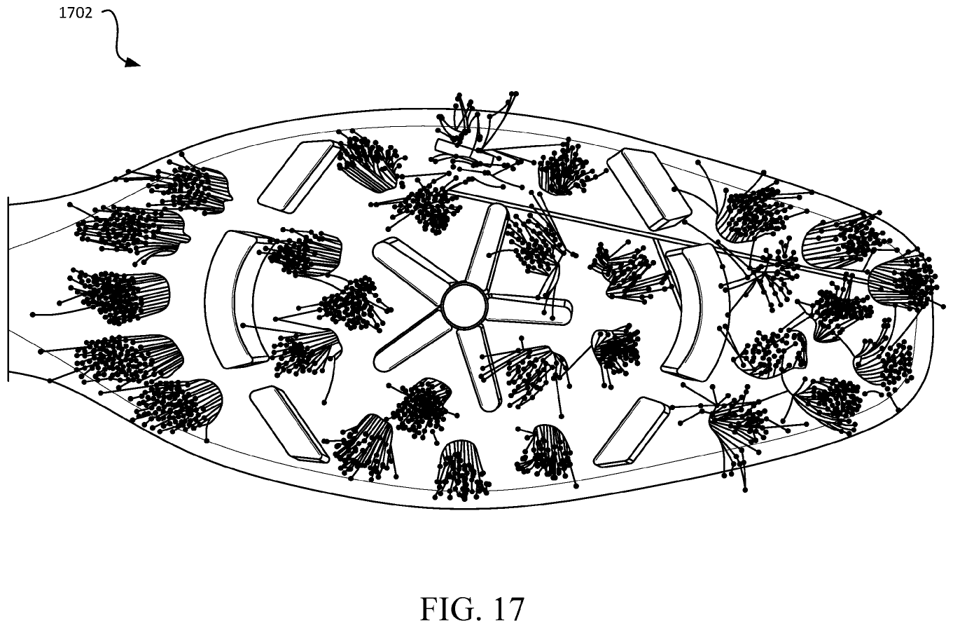

In one or more scenarios, the one or more digital image frames of the toothbrush may be communicated to a composite imaging generator 616 to develop one or more composite images of the toothbrush 604. FIG. 17 illustrates an example composite image 1702 that may be constructed from the captured one or more digital images of the toothbrush 604 on the conveyor 606. In one or more scenarios, one or more quality control inspections may be conducted automatically, perhaps in substantially real time, based, at least in part, on the one or more captured digital image frames and/or the one or more composite images, for example. For example, filament top defects, tufting defects, anchor imperfections, and/or loss tufts, among other quality control deviations may be detected based the composite image 1702 and/or the one or more captured digital image frames of FIG. 16.

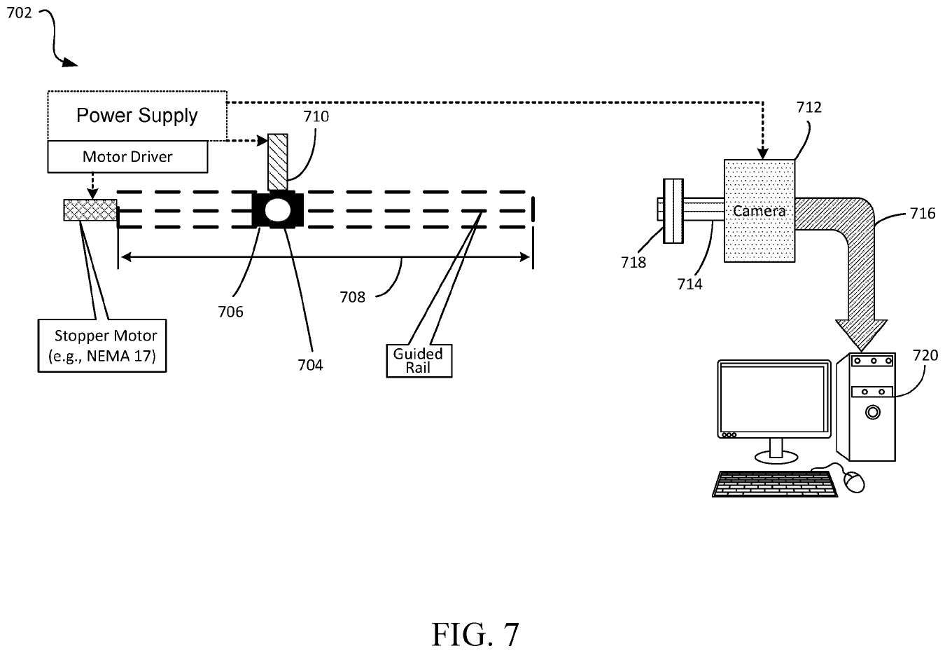

FIG. 7 illustrates an example of a kinetic imaging technique arrangement 702 for a product during a manufacturing process. FIG. 7 illustrates a somewhat more granular arrangement of the kinetic imaging technique than that illustrated in FIG. 6. In FIG. 7, a toothbrush 704 (e.g., in an upward/vertical orientation, in a flat/horizontal orientation, and/or in an angled/intermediate orientation) may be conveyed on a line on a slider 706 on approximately 500 millimeters (mm) of a conveyor line 708 (e.g., curved). A product position sensor/trigger (e.g., optical/electrical switch) 710 may indicate the toothbrush 704 has entered an imaging area/zone of the conveyor line 708. A camera device 712 (e.g., industrial camera, 9MP, 95 fps, with a fixed focus lens 714) may be controlled to capture one or more digital image frames of the toothbrush 704. The captured one or more digital image frames 716 (e.g., an image “stack”, like the image frames of FIG. 16) may be communicated to a composite image generator 720 for construction of one or more composite images (e.g., like composite image 1702). In one or more scenarios, an LED illuminator 718 may be used in the capture of the one or more digital image frames of the toothbrush 704. The LED illuminator 718 may be controlled to provide a determined illumination, such as LED light intensity and/or LED light color, for example.

In one or more scenarios, one or more toothbrush 704 head moving postures may be accommodated by the kinetic imaging technique, such as angle(s) of the toothbrush 704 head to face the camera lens and/or the speed relative to camera lens, for example. One or more camera device 712 control parameters may be considered/determined such as imagining conditions, (e.g., best) shutter speed(s), (e.g., best) imaging intervals, and/or other control parameters, for example.

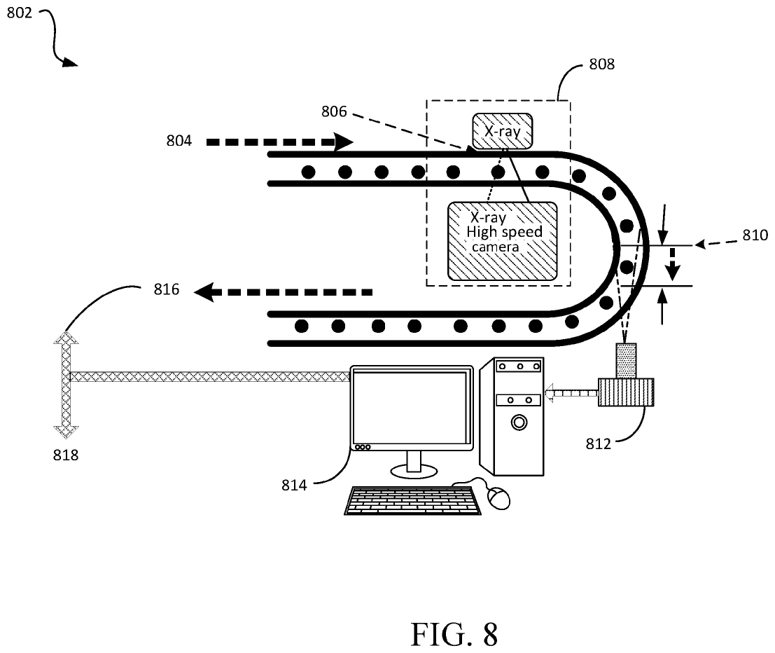

FIG. 8 illustrates an example of a kinetic imaging technique arrangement 802 for a product during a manufacturing process. FIG. 8 illustrates a somewhat broader arrangement of the kinetic imaging technique than that illustrated in FIG. 6. At 804, at a product loading station, the product (e.g., a toothbrush) may be positioned in an upward/vertical orientation, a flat/horizontal orientation, and/or an angled/intermediate orientation with the bristle tip(s) facing the conveyor moving direction. At 806, a (e.g., first) product position sensor may trigger an X-Ray camera imaging process. At 808, as the toothbrush moves into position, the X-ray camera may take one or more images for (e.g., each) toothbrush, perhaps for example for anchor depth profiling and/or bristle side view profiling quality control inspections.

At 810, a (e.g., “second”) product position sensor/trigger may indicate toothbrush has entered an imaging area. At 812, perhaps as triggered by the second product position sensor, for example, a “vision” camera device (e.g., by Olympus, by Bauer Full HD 95 FPS, or the like) may be controlled to capture one or more (e.g., 10 or more) digital image frames of the toothbrush while the toothbrush is within the imaging area/zone. For example, the camera device may capture at least ten or more digital image frames that may (e.g., respectively) be focused on different bristle profiles/depths and/or different regions of the toothbrush. The one or more kinetically captured digital image frames may be used for end rounding and/or bristle pattern quality control inspections, among other quality inspections.

In one or more scenarios, at 814 a composite image/image fusion generator may be used to create one or more composite images of the toothbrush. The composite image generator may be used for quality control measurements/assessments. The composite image generator may utilize artificial intelligence/machine learning algorithms to aid imaging analysis, and for quality control reporting, product and/or manufacturing line maintenance warnings, and/or manufacturing line control, or the like. At 816, the toothbrush may receive a “pass or fail” quality control assessment at a product accept/reject station, for example. At 818, the manufacturing control process may receive quality control warnings/early warnings and/or maintenance warnings, perhaps based, at least in part, on quality control assessments (e.g., end rounding and/or bristle profiling assessments, among other assessments).

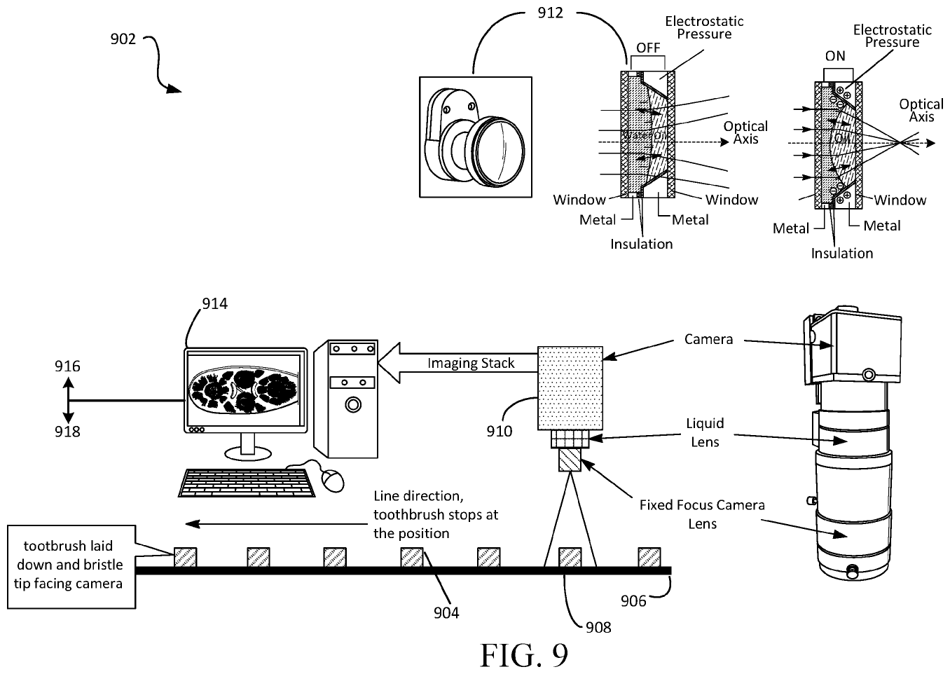

FIG. 9 illustrates an example of a kinetic imaging technique arrangement 902 for a product during a manufacturing process. A product 904 (e.g., a toothbrush) may travel in the manufacturing process on conveyor line 906. The toothbrush 904 arrival in an imaging area/zone 908 may be detected by a product position sensor (not shown), or other product position detection techniques. Camera device 910 may be controlled to capture one or more digital image frames of the toothbrush 904, perhaps for example while the toothbrush 904 remains in the imaging area 908.

In one or more scenarios, camera device 910 may comprise a fixed focus lens and/or a liquid lens 912. The liquid lens 912 (e.g., with a response time of 10 ms or the like) may be a fast electrically tunable lens that may have a response time, rise time, and/or settling time of approximately ten milliseconds (10 ms), for example. The toothbrush 904 may be stopped/stationary, or moving at a substantially reduced conveyor line 906 speed/velocity, perhaps relative to other sections of the conveyor 906, during the digital imaging frame capture, for example. The camera device 910 may be controlled to utilize the liquid lens 912 to adjust the focus point (e.g., respectively) for one or more of the captured digital image frames at (e.g., respectively) different profile depths and/or different regions of the toothbrush 904. The one or more kinetically captured digital image frames may be used for end rounding and/or bristle pattern quality control inspections, among other quality inspections.

In one or more scenarios, toothbrush 904 may be in an “upward” or “standing” or vertical orientation, a “laid down” or “flat” or horizontal orientation, and/or an “angled” or “slanted” or “leaning” or intermediate orientation, perhaps for example with the toothbrush 904 bristle tip (not shown) facing the camera device 910.

In one or more scenarios, at 914 a composite image/image fusion generator may be used to create one or more composite images of the toothbrush 904. The composite image generator may be used for quality control measurements/assessments. The composite image generator may utilize artificial intelligence/machine learning algorithms to aid imaging analysis, and for quality control reporting, product and/or manufacturing line maintenance warnings, and/or manufacturing line control, or the like. At 916, the toothbrush 904 may receive a “pass or fail” quality control assessment at a product accept/reject station, for example. At 918, the manufacturing control process may receive quality control warnings/early warnings and/or maintenance warnings, perhaps based, at least in part, on quality control assessments (e.g., end rounding and/or bristle profiling assessments, among other assessments).

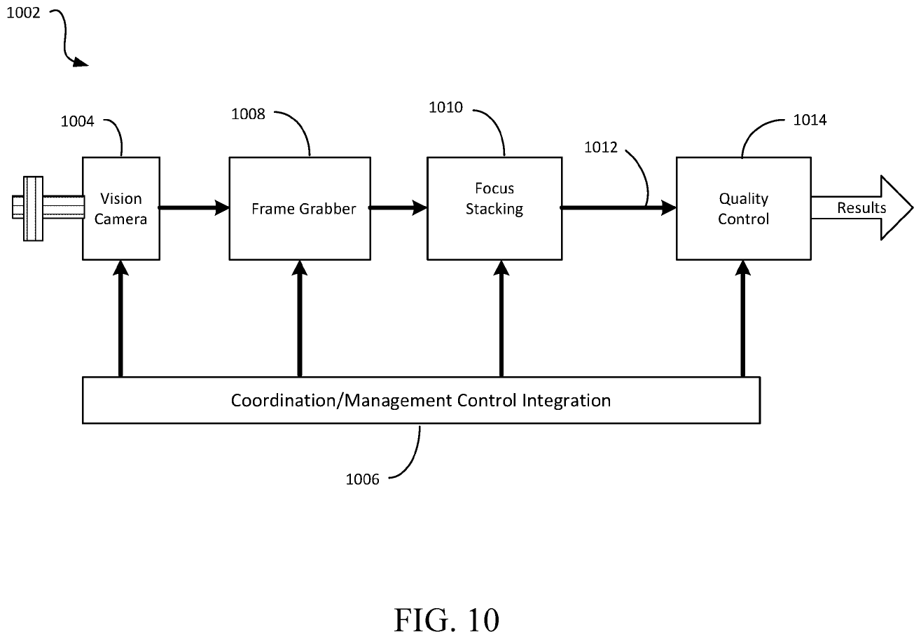

FIG. 10 is an example block diagram of a kinetic imaging technique integrated into an extended manufacturing quality control scheme. FIG. 10 illustrates an integrated manufacturing quality control scheme 1002. As described herein, one or more digital image frames may be captured of a product in a manufacturing process by a (e.g., “vision”) camera device 1004. One or more manufacturing control devices 1006 may control/coordinate at least some of the operations of the respective components of the integrated manufacturing quality control scheme 1002.

In one or more scenarios, a frame grabber 1008 (e.g., a 10 GigE device, and/or a USB 3.0 device, or the like) may convey the captured one or more digital image frames of the product (e.g., a stack of digital image frames) from the camera device 1004 to a composite image generator 1010. The composite image generator 1010 may construct/generate one or more composite images 1012 of the product based, at least in part, on the one or more captured digital image frames/stack. The one or more composite images 1012 may be communicated to a quality control module 1014 that may use machine learning/artificial intelligence quality control schemes, statistical quality control techniques, and/or analytical quality control tools to generate one or more quality control assessment results and/or manufacturing control settings (e.g., pass/fail, manufacturing variation early warnings, or the like).

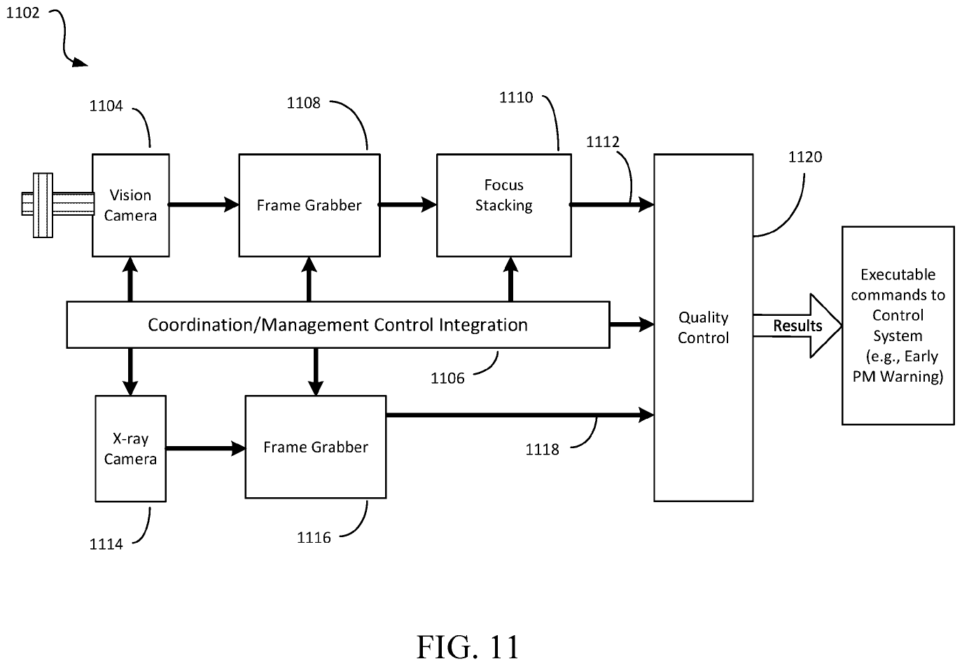

FIG. 11 is an example block diagram of a kinetic imaging technique integrated into an extended manufacturing quality control scheme. FIG. 11 illustrates an integrated manufacturing quality control scheme 1102. As described herein, one or more digital image frames may be captured of a product in a manufacturing process by a (e.g., “vision”) camera device 1104. One or more manufacturing control devices 1106 may control/coordinate at least some of the operations of the respective components of the integrated manufacturing quality control scheme 1102.

In one or more scenarios, a frame grabber 1108 (e.g., a 10 GigE device, and/or a USB 3.0 device, or the like) may convey the captured one or more digital image frames of the product (e.g., a stack of digital image frames) from the camera device 1104 to a composite image generator 1110. The composite image generator 1110 may construct/generate one or more composite images 1112 of the product based, at least in part, on the one or more captured digital image frames/stack. The one or more composite images 1112 may be communicated to a quality control module 1120.

As described herein, one or more X-ray image frames may be captured of a product in a manufacturing process by an X-ray camera device 1114. An X-ray Camera Adapter and Frame Grabber 1116 may convey the captured one or more X-ray image frames 1118 of the product from the X-ray camera device 1114 to the quality control module 1120.

The quality control module 1120 may use machine learning/artificial intelligence quality control schemes, statistical quality control techniques, and/or analytical quality control tools to generate one or more quality control assessment results and/or manufacturing control settings (e.g., pass/fail, manufacturing variation early warnings, or the like).

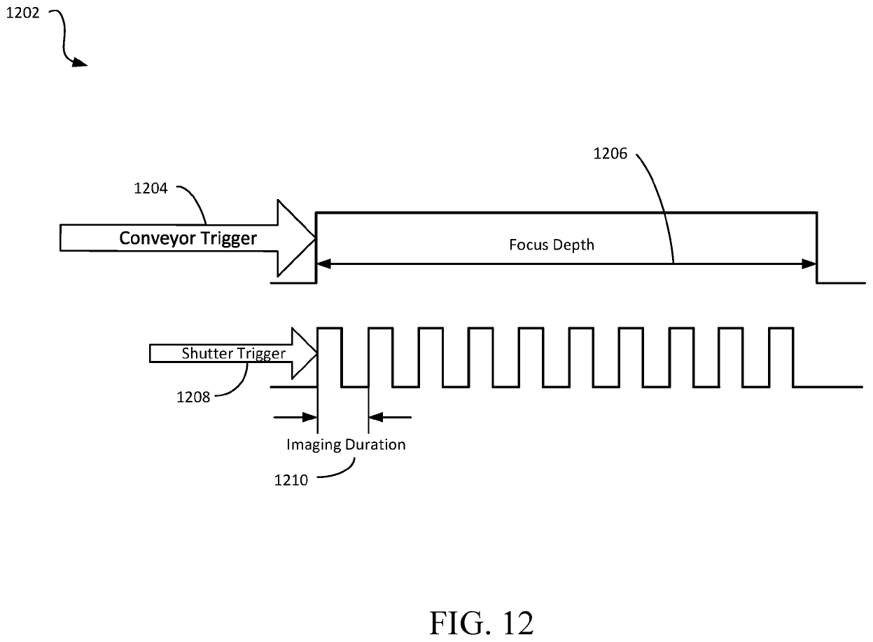

FIG. 12 is an example illustration of a camera device timing/parameter sequence/pattern for the capture of one or more digital image frames of a product during a manufacturing process. In one or more scenarios, perhaps for example in manufacturing scenarios in which the product (e.g., a toothbrush) may be in a vertical orientation, a horizontal orientation, and/or an intermediate orientation on a conveyor line (not shown), a (e.g., vision) camera device (not shown, e.g., with a fixed focus lens and/or a liquid lens) may be controlled such that it captures one or more digital image frames of the product. The control of the camera device imaging may include determination, calculation, acquisition, setting, and/or adjustment of one or more camera device parameters and/or manufacturing process parameters, such as focus depth, conveyor speed/velocity, one or more (e.g., each) image/imaging duration, a number of digital image frames to be captured, and/or shutter trigger sequence, among other parameters.

In an example camera device timing/parameter sequence/pattern 1202, a process control device/logic controller (not shown) may detect a signal from a product position sensor (not shown) at 1204 and may determine a focus depth for the camera device, such as Focus Depth 1206, where Focus Depth=Vconveyor*Number of Images, for example.

In one or more scenarios, a shutter trigger sequence 1208 may be determined that may be used to control the camera device in the capture of the one or more digital image frames, for example. An image/imaging duration 1210 may be determined and/or calculated, which may be used to control the camera device in the capture of the one or more digital image frames of the product, for example.

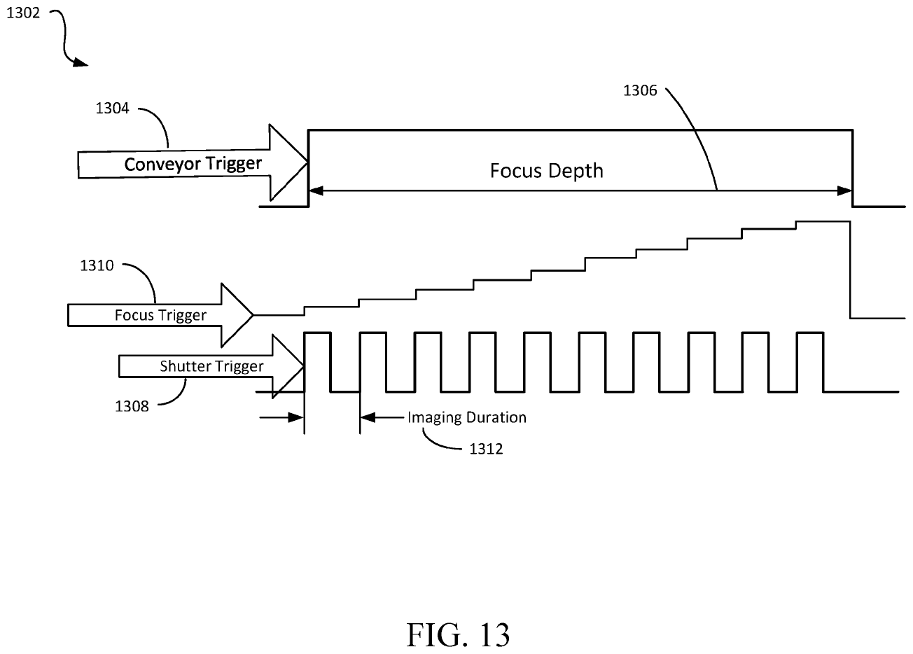

FIG. 13 is an example illustration of a camera device timing/parameter sequence/pattern for the capture of one or more digital image frames of a product during a manufacturing process. In one or more scenarios, perhaps for example in manufacturing scenarios in which the product (e.g., a toothbrush) may be in a vertical orientation, a horizontal orientation, and/or an intermediate orientation on a conveyor line (not shown), a (e.g., vision) camera device (not shown, e.g., with a fixed focus lens and/or a liquid lens) may be controlled such that it captures one or more digital image frames of the product. The control of the camera device imaging may include determination, calculation, acquisition, setting, and/or adjustment of one or more camera device parameters and/or manufacturing process parameters, such as focus depth, total image/imaging duration, one or more (e.g., each) focus rise/rising time, one or more (e.g., each) image/imaging duration, a number of digital image frames to be captured, a focus trigger sequence, and/or shutter trigger sequence, among other parameters.

In an example camera device timing/parameter sequence/pattern 1302, a process control device/logic controller (not shown) may detect a signal from a product position sensor (not shown) at 1304 and may determine a focus depth for the camera device, such as Focus Depth 1306, where Focus Depth=Tfocus*Number of Images, for example.

In one or more scenarios, a shutter trigger sequence 1308 may be determined that may be used to control the camera device in the capture of the one or more digital image frames, for example. A focus trigger sequence 1310 may be determined that may be used to control the camera device in the capture of the one or more digital image frames, for example. An image/imaging duration 1312 may be determined and/or calculated, which may be used to control the camera device in the capture of the one or more digital image frames of the product, for example.

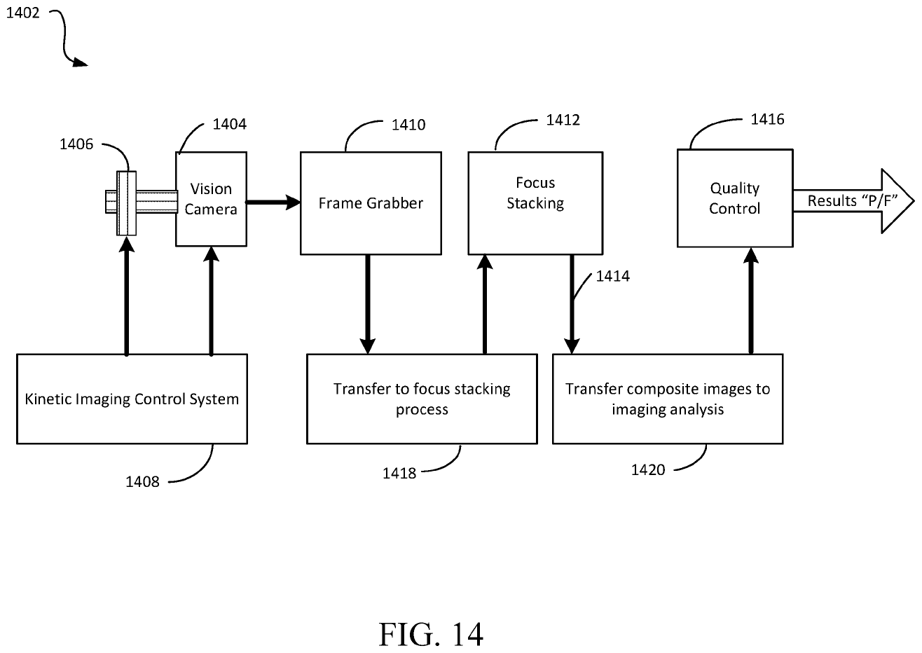

FIG. 14 is an example block diagram of a kinetic imaging technique integrated into an extended manufacturing quality control scheme. FIG. 14 illustrates an integrated manufacturing quality control scheme 1402. As described herein, one or more digital image frames may be captured of a product in a manufacturing process by a (e.g., “vision”) camera device 1404. An LED illuminator 1406 may be controlled to provide sufficient light for the capture of the one or more digital image frames. A manufacturing control device 1408 may control/coordinate at least some of the operations of the camera device 1404 and/or the LED illuminator 1406.

In one or more scenarios, a frame grabber/storage 1410 (e.g., a 10 GigE device, and/or a USB 3.0 device, or the like) may convey the captured one or more digital image frames of the product (e.g., a stack of digital image frames) from the camera device 1404 to a composite image generator 1412. In one or more scenarios, at 1418, the captured one or more digital image frames (e.g., stack of image frames) may be automatically and/or r manually transferred/communicated from the frame grabber 1410 to the composite image generator 1412.

The composite image generator 1412 may construct/generate one or more composite images 1414 of the product based, at least in part, on the one or more captured digital image frames/stack. The one or more composite images 1414 may be communicated to a quality control module 1416 that may use machine learning/artificial intelligence quality control schemes, statistical quality control techniques, and/or analytical quality control tools to generate one or more quality control assessment results and/or manufacturing control settings (e.g., pass/fail, manufacturing variation early warnings, or the like). In one or more scenarios, at 1420, the one or more composite images 1414 (e.g., Z-mapping images, etc.) may be manually and/or automatically transferred/communicated from the composite image generator 1412 to the quality control module 1416.

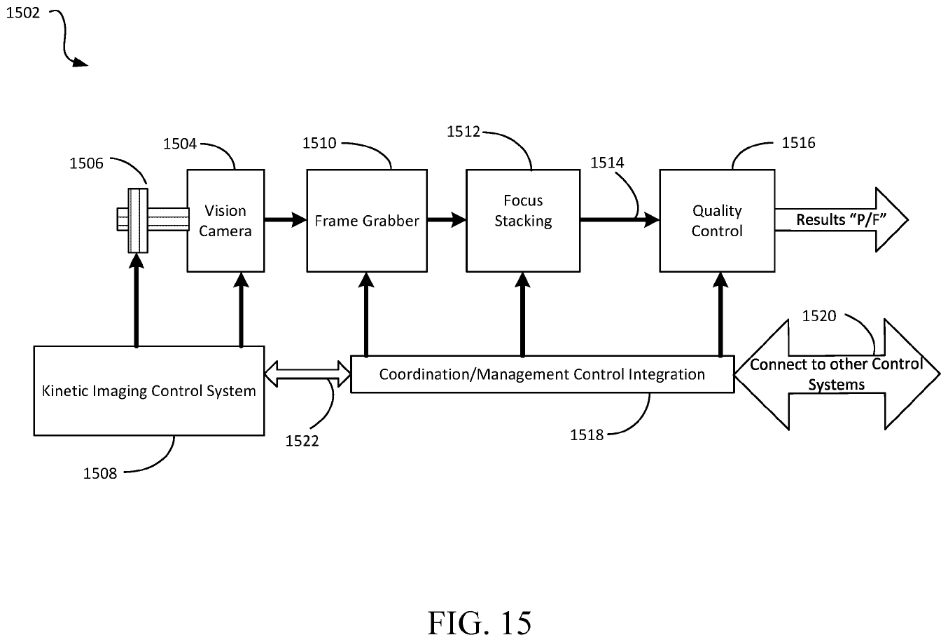

FIG. 15 is an example block diagram of a kinetic imaging technique integrated into an extended manufacturing quality control scheme. FIG. 15 illustrates an integrated manufacturing quality control scheme 1502. As described herein, one or more digital image frames may be captured of a product in a manufacturing process by a (e.g., “vision”) camera device 1504. An LED illuminator 1506 may be controlled to provide sufficient light for the capture of the one or more digital image frames. A manufacturing control device 1508 may control/coordinate at least some of the operations of the camera device 1504 and/or the LED illuminator 1506.

In one or more scenarios, a frame grabber/storage 1510 (e.g., a 10 GigE device, and/or a USB 3.0 device, or the like) may convey the captured one or more digital image frames of the product (e.g., a stack of digital image frames) from the camera device 1504 to a composite image generator 1512.

The composite image generator 1512 may construct/generate one or more composite images 1514 of the product based, at least in part, on the one or more captured digital image frames/stack. The one or more composite images 1514 may be communicated to a quality control module 1516 that may use machine learning/artificial intelligence quality control schemes, statistical quality control techniques, and/or analytical quality control tools to generate one or more quality control assessment results and/or manufacturing control settings (e.g., pass/fail, manufacturing variation early warnings, or the like).

In one or more scenarios, one or more manufacturing control devices 1518 may control/coordinate at least some of the operations of the respective components of the integrated manufacturing quality control scheme 1502, such as the frame grabber 1510, the composite image generator 1512, and/or the quality control module 1516, for example. The one or more manufacturing control devices 1518 may communicate with one or more manufacturing and/or enterprise data management systems 1520, among other systems, for example.

The one or more manufacturing devices 1518 may, at 1522, communicate one or more manufacturing process control parameters (e.g., camera device 1504 parameters, and/or product stock keeping unit (SKU), etc.) with the manufacturing control device 1508.

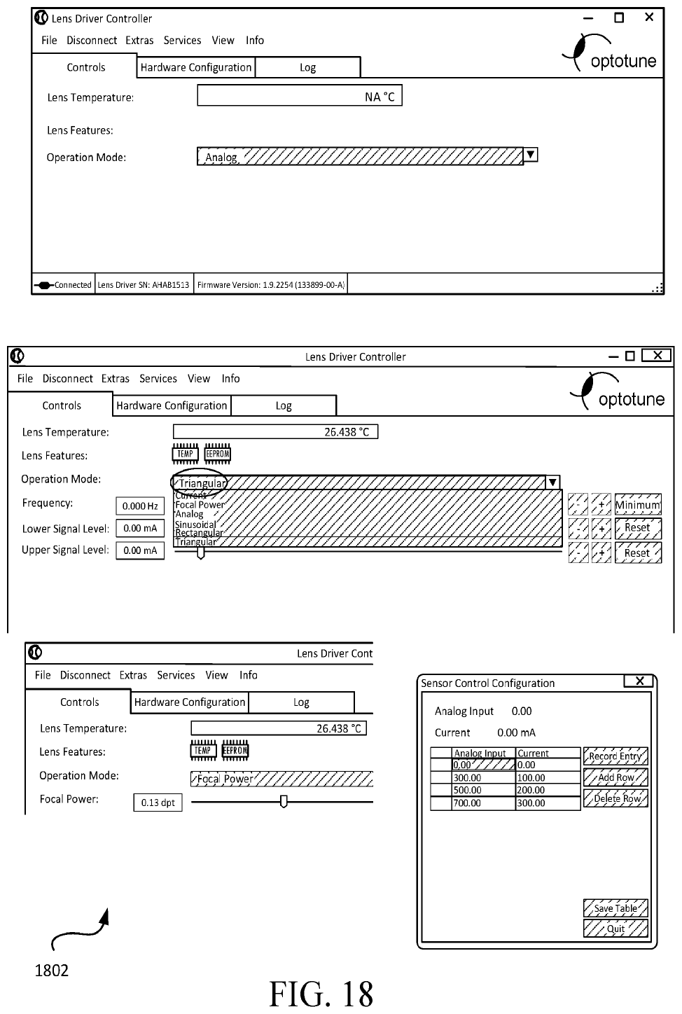

FIG. 18 illustrates an example a camera device liquid lens control configuration and control technique. FIG. 18 illustrates a plurality of graphical user interfaces (GUI) 1802 that may be used for configuration and/or control of a liquid lens (not shown) in the capture of one or more digital image frames of a product during a manufacturing process.

In one or more scenarios, a liquid lens controller housing (not shown) may be opened and/or an analog input “B” (e.g., 0˜5V) may be located. A ten (10) bit digital/analog (D/A) converter output may be connected to the analog input “B”, for example.

In a Liquid Lens Controller “Hardware Configuration” Page (for example), one or more of the following parameters may be set: “Max Current” to 292.84 mA, “Lower Software Limit” to −99.97 mA, and/or “Upper Software Limit” to 149.96 mA, for example.

In a Lens Controller “Controls” Page (for example), an “Operation Mode” may be set to Analog. Analog input “A” may serve as a “Trigger output” to trigger a camera device for one or more digital image frame capture.

In one or more scenarios, at least one lookup table for voltage versus power may be built. In a “Focus Power” mode, a slider may be adjusted to get at least two focus point power numbers, for example. In an “Analog” mode, an input voltage from 0 to 5V may be set. The voltages for the at least two focus points may be obtained and/or recorded. In a “Current” or “Focal” Power mode, under “Services,” a “Sensor Control” may be selected. In/under an “Extras” mode/menu, “Sensor Control Configuration” may be selected. The voltage and power values may be added to the table and/or the table may be saved. The lens controller may be run/operated in an “Analog” mode, for example.

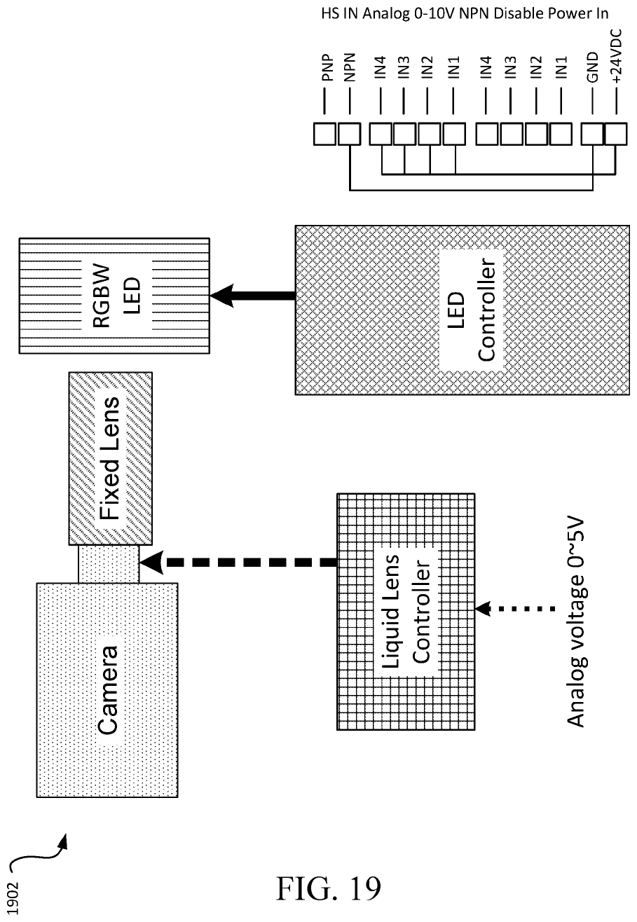

FIG. 19 is an example diagram of at least one technique for digital image frame capture light control. The camera vision light control arrangement 1902 illustrates a general arrangement of a camera device comprising a fixed focus lens and a liquid lens with a light source LED (e.g., a RGB White LED). The LED controller may control a brightness of the RGBW LED to facilitate the capture of the one or more digital image frames of a product in a manufacturing process. For example, proper LED illumination may enhance the image detail such that at least some of the captured digital image frames may (e.g., each) better represent a (e.g., respectively) different focused depth profile of the product, and/or a (e.g., respectively) different focused region of the product.

Those skilled in the art will appreciate that the subject matter described herein may at least facilitate a “hands off” and/or high-speed quality control assessment of one or more product quality parameters while the product is in the manufacturing environment. For example, perhaps instead of having a human quality control technician either physically inspecting the product by hand, and/or removing the product from a conveyor line during manufacture for such inspection, the one or more quality control assessments may be made based on one or more image frames captured during the manufacturing process. The one or more image frames may capture a respectively different focused depth profile of the product, and/or a respectively different focused region of the product. Quality control assessments, perhaps culminating in a “pass or fail”, or “recycle”, “rework”, “pass on to the next phase of manufacturing”, and/or “pass on to packaging and/or distribution”, or the like, may be based on the one or more image frames of the product and/or a composite image of the product constructed from the one or more image frames.

The subject matter of this disclosure, and components thereof, can be realized by instructions that upon execution cause one or more processing devices to carry out the processes and/or functions described herein. Such instructions can, for example, comprise interpreted instructions, such as script instructions, e.g., JavaScript or ECMAScript instructions, or executable code, and/or other instructions stored in a computer readable medium.

Implementations of the subject matter and/or the functional operations described in this specification and/or the accompanying figures can be provided in digital electronic circuitry, in computer software, firmware, and/or hardware, including the structures disclosed in this specification and their structural equivalents, and/or in combinations of one or more of them. The subject matter described in this specification can be implemented as one or more computer program products, e.g., one or more modules of computer program instructions encoded on a tangible program carrier for execution by, and/or to control the operation of, data processing apparatus.

A computer program (also known as a program, software, software application, script, or code) can be written in any form of programming language, including compiled or interpreted languages, and/or declarative or procedural languages. It can be deployed in any form, including as a stand-alone program or as a module, component, subroutine, and/or other unit suitable for use in a computing environment. A computer program may or might not correspond to a file in a file system. A program can be stored in a portion of a file that holds other programs and/or data (e.g., one or more scripts stored in a markup language document), in a single file dedicated to the program in question, and/or in multiple coordinated files (e.g., files that store one or more modules, sub programs, or portions of code). A computer program can be deployed to be executed on one computer or on multiple computers that may be located at one site or distributed across multiple sites and/or interconnected by a communication network.

The processes and/or logic flows described in this specification and/or in the accompanying figures may be performed by one or more programmable processors executing one or more computer programs to perform functions by operating on input data and/or generating output, thereby tying the process to a particular machine (e.g., a machine programmed to perform the processes described herein). The processes and/or logic flows can also be performed by, and apparatus can also be implemented as, special purpose logic circuitry, e.g., an FPGA (field programmable gate array) and/or an ASIC (application specific integrated circuit).

Computer readable media suitable for storing computer program instructions and/or data may include all forms of non-volatile memory, media and memory devices, including by way of example semiconductor memory devices (e.g., EPROM, EEPROM, and/or flash memory devices); magnetic disks (e.g., internal hard disks or removable disks); magneto optical disks; and/or CD ROM and DVD ROM disks. The processor and/or the memory can be supplemented by, or incorporated in, special purpose logic circuitry.

While this specification and the accompanying figures contain many specific implementation details, these should not be construed as limitations on the scope of any invention and/or of what may be claimed, but rather as descriptions of features that may be specific to described example implementations. Certain features that are described in this specification in the context of separate implementations can also be implemented in combination in perhaps one implementation. Various features that are described in the context of perhaps one implementation can also be implemented in multiple combinations separately or in any suitable sub-combination. Although features may be described above as acting in certain combinations and/or perhaps even (e.g., initially) claimed as such, one or more features from a claimed combination can in some cases be excised from the combination. The claimed combination may be directed to a sub-combination and/or variation of a sub-combination.

While operations may be depicted in the drawings in an order, this should not be understood as requiring that such operations be performed in the particular order shown and/or in sequential order, and/or that all illustrated operations be performed, to achieve useful outcomes. The described program components and/or systems can generally be integrated together in a single software product and/or packaged into multiple software products.

Examples of the subject matter described in this specification have been described. The actions recited in the claims can be performed in a different order and still achieve useful outcomes, unless expressly noted otherwise. For example, the processes depicted in the accompanying figures do not require the particular order shown, and/or sequential order, to achieve useful outcomes. Multitasking and parallel processing may be advantageous in one or more scenarios.

While the present disclosure has been illustrated and described in detail in the drawings and foregoing description, the same is to be considered as illustrative and not restrictive in character, it being understood that only certain examples have been shown and described, and that all changes and modifications that come within the spirit of the present disclosure are desired to be protected

### Exemplary Claim Set

Exemplary claim 1. A method of controlling a capture of one or more digital image frames of a product on a conveyor line, the method performed by a manufacturing control device, the manufacturing control device being in communication with at least one camera, the method comprising: receiving, by the manufacturing control device, a product position trigger signal indicating that the product has entered an imaging zone of the conveyor line; determining, by the manufacturing control device, a shutter trigger sequence for the at least one camera such that at least a predetermined number of the digital image frames are captured while the product is within the imaging zone of the conveyor line; and controlling, by the manufacturing control device, the capture of at least the predetermined number of the digital image frames via the at least one camera using the determined shutter trigger sequence upon receipt of the product position signal.

Exemplary claim 2. The method of exemplary claim 1, wherein the determining the shutter trigger sequence for the at least one camera further comprises: determining, by the manufacturing control device, an image frame duration for each of the predetermined number of the digital image frames.

Exemplary claim 3. The method of any of the previous exemplary claims, wherein the determining the shutter trigger sequence for the at least one camera is based, at least in part, on at least one image frame rate of the at least one camera.

Exemplary claim 4. The method of any of the previous exemplary claims, wherein the at least one camera comprises a fixed focus lens.

Exemplary claim 5. The method of exemplary claim 4, wherein the product is disposed on the conveyor line in at least one of: a vertical orientation, a horizontal orientation, or an intermediate orientation, the method further comprising: receiving, by the manufacturing control device, a conveyor line speed, wherein the determining the shutter trigger sequence for the at least one camera is based, at least in part, on the conveyor line speed.

Exemplary claim 6. The method of exemplary claim 4, wherein the at least one camera further comprises a liquid lens, the liquid lens having a focus rise time, the method further comprising: determining, by the manufacturing control device, a focus trigger sequence for the at least one camera such that the at least predetermined number of the digital image frames are captured at one or more focus points while the product is within the imaging zone of the conveyor line, wherein the determining the shutter trigger sequence for the at least one camera is based, at least in part, on the focus rise time.

Exemplary claim 7. The method of any of the previous exemplary claims, wherein the product is disposed on the conveyor line in at least one of: a vertical orientation, a horizontal orientation, or an intermediate orientation, and the conveyor line is stopped during the capture of the at least predetermined number of the digital image frames via the at least one camera.

Exemplary claim 8. The method of any of the previous exemplary claims, wherein the product is at least one of: a toothbrush, a hairbrush, or a non-round hairbrush.

Exemplary claim 9. The method of any of the previous exemplary claims, wherein the captured digital image frames are at least one of: one or more digital still images, or one or more digital image frames of a digital video stream.

Exemplary claim 10. The method of any of the previous exemplary claims, wherein the determined the shutter trigger sequence controls the at least one camera such that at least some of the captured digital image frames each represent at least one of: a respectively different focused depth profile of the product, or a respectively different focused region of the product.

Exemplary claim 11. A manufacturing control device configured to control a capture of one or more digital image frames of a product on a conveyor line, the manufacturing control device being in communication with at least one camera, the manufacturing control device comprising: a memory; and a processor, the processor configured at least to: receive a product position trigger signal indicating that the product has entered an imaging zone of the conveyor line; determine a shutter trigger sequence for the at least one camera such that at least a predetermined number of the digital image frames are captured while the product is within the imaging zone of the conveyor line; and control the capture of at least the predetermined number of the digital image frames via the at least one camera using the determined shutter trigger sequence upon receipt of the product position signal.

Exemplary Claim The device of claim 11, wherein the processor is further configured to: determine an image frame duration for each of the predetermined number of the digital image frames; and use the determined image frame duration for the determination of the shutter trigger sequence for the at least one camera.

Exemplary claim 13. The device of any of exemplary claims 11 to 12, wherein the processor is further configured such that the determination of the shutter trigger sequence for the at least one camera is based, at least in part, on at least one image frame rate of the at least one camera.

Exemplary claim 14. The device of any of exemplary claims 11 to 13, wherein the at least one camera comprises a fixed focus lens.

Exemplary claim 15. The device of exemplary claim 14, wherein the product is disposed on the conveyor line in at least one of: a vertical orientation, a horizontal orientation, or an intermediate orientation, the processor being further configured to: receive a conveyor line speed, wherein the processor is further configured such that the determination of the shutter trigger sequence for the at least one camera is based, at least in part, on the conveyor line speed.

Exemplary claim 16. The device of exemplary claim 14, wherein the at least one camera further comprises a liquid lens, the liquid lens having a focus rise time, the processor being further configured to: determine a focus trigger sequence for the at least one camera such that the at least predetermined number of the digital image frames are captured at one or more focus points while the product is within the imaging zone of the conveyor line, wherein the processor is further configured such that the determination of the shutter trigger sequence for the at least one camera is based, at least in part, on the focus rise time.

Exemplary claim 17. The device of any of exemplary claims 11 to 16, wherein the product is disposed on the conveyor line in at least one of: a vertical orientation, a horizontal orientation, or an intermediate orientation, and the conveyor line is stopped during the capture of the at least predetermined number of the digital image frames via the at least one camera.

Exemplary claim 18. The device of any of exemplary claims 11 to 17, wherein the product is at least one of: a toothbrush, a hairbrush, or a non-round hairbrush.

Exemplary claim 19. The device of any of exemplary claims 11 to 18, wherein the captured digital image frames arc at least one of: one or more digital still images, or one or more digital image frames of a digital video stream.

Exemplary claim 20. The device of any of exemplary claims 11 to 19, wherein the processor is further configured such that the determined shutter trigger sequence controls the at least one camera such that at least some of the captured digital image frames each represent at least one of: a respectively different focused depth profile of the product, or a respectively different focused region of the product.

Exemplary claim 21. A manufacturing control system configured to control a capture of one or more digital image frames of a product on a conveyor line, the system comprising: a camera; a position detector; and a control device, comprising: a memory; and a processor, the processor configured at least to: receive a product position trigger signal from the position detector indicating that the product has entered an imaging zone of the conveyor line; determine a shutter trigger sequence for the camera such that at least a predetermined number of the digital image frames are captured while the product is within the imaging zone of the conveyor line; and control the capture of at least the predetermined number of the digital image frames via the camera using the determined shutter trigger sequence upon receipt of the product position signal.

Exemplary claim 22. The system of exemplary claim 21, wherein the processor is further configured to: determine an image frame duration for each of the predetermined number of the digital image frames; and use the determined image frame duration for the determination of the shutter trigger sequence for the camera.

Exemplary claim 23. The system of any of exemplary claims 21 to 22, wherein the processor is further configured such that the determination of the shutter trigger sequence for the camera is based, at least in part, on at least one image frame rate of the camera.

Exemplary claim 24. The system of any of exemplary claims 21 to 23, wherein the camera comprises a fixed focus lens.

Exemplary claim 25. The system of exemplary claim 24, wherein the product is disposed on the conveyor line in at least one of: a vertical orientation, a horizontal orientation, or an intermediate orientation, the processor being further configured to: receive a conveyor line speed, wherein the processor is further configured such that the determination of the shutter trigger sequence for the camera is based, at least in part, on the conveyor line speed.

Exemplary claim 26. The system of exemplary claim 24, wherein the camera further comprises a liquid lens, the liquid lens having a focus rise time, the processor being further configured to: determine a focus trigger sequence for the at least one camera such that the at least predetermined number of the digital image frames are captured at one or more focus points while the product is within the imaging zone of the conveyor line, wherein the processor is further configured such that the determination of the shutter trigger sequence for the at least one camera is based, at least in part, on the focus rise time.

Exemplary claim 27. The system of any of exemplary claims 21 to 26, wherein the product is disposed on the conveyor line in at least one of: a vertical orientation, a horizontal orientation, or an intermediate orientation, and the conveyor line is stopped during the capture of the at least predetermined number of the digital image frames via the at least one camera.

Exemplary claim 28. The system of any of exemplary claims 21 to 27, wherein the product is at least one of: a toothbrush, a hairbrush, or a non-round hairbrush.

Exemplary claim 29. The system of any of exemplary claims 21 to 28, wherein the captured digital image frames are at least one of: one or more digital still images, or one or more digital image frames of a digital video stream.

Exemplary claim 30. The system of any of exemplary claims 21 to 29, wherein the processor is further configured such that the determined shutter trigger sequence controls the camera such that at least some of the captured digital image frames each represent at least one of: a respectively different focused depth profile of the product, or a respectively different focused region of the product.

Exemplary claim 31. A method to control a capture of one or more digital image frames of a product on a conveyor line performed by a manufacturing control system, the method comprising: detecting, by a position detector, that the product has entered an imaging zone of the conveyor line; sending, by the position detector, a product position trigger signal indicating that the product has entered the imaging zone of the conveyor line; receiving, by a control device, the product position trigger signal from the position detector; determining, by the control device, a shutter trigger sequence for the camera such that at least a predetermined number of the digital image frames are captured while the product is within the imaging zone of the conveyor line; controlling, by the control device, the capture of at least the predetermined number of the digital image frames via the camera using the determined shutter trigger sequence upon receipt of the product position signal; and capturing, by a camera, at least the predetermined number of the digital image frames per the determined shutter trigger sequence.

Exemplary claim 32. The method of exemplary claim 31, wherein the determining the shutter trigger sequence for the camera further comprises: determining, by the control device, an image frame duration for each of the predetermined number of the digital image frames.

Exemplary claim 33. The method of any of exemplary claims 31 to 32, wherein the determining the shutter trigger sequence for the camera is based, at least in part, on at least one image frame rate of the camera.

Exemplary claim 34. The method of any of exemplary claims 31 to 33, wherein the camera comprises a fixed focus lens.

Exemplary claim 35. The method of exemplary claim 34, wherein the product is disposed on the conveyor line in at least one of: a vertical orientation, a horizontal orientation, or an intermediate orientation, the method further comprising: receiving, by the control device, a conveyor line speed, wherein the determining the shutter trigger sequence for the camera is based, at least in part, on the conveyor line speed.

Exemplary Claim 36. The method of exemplary claim 34, wherein the camera further comprises a liquid lens, the liquid lens having a focus rise time, the method further comprising: determining, by the control device, a focus trigger sequence for the at least one camera such that the at least predetermined number of the digital image frames are captured at one or more focus points while the product is within the imaging zone of the conveyor line, wherein the determining the shutter trigger sequence for one camera is based, at least in part, on the focus rise time.

Exemplary claim 37. The method of any of exemplary claims 31 to 36, wherein the product is disposed on the conveyor line in at least one of: a vertical orientation, a horizontal orientation, or an intermediate orientation, and the conveyor line is stopped during the capture of the at least predetermined number of the digital image frames via the camera.

Exemplary claim 38. The method of any of exemplary claims 31 to 37, wherein the product is at least one of: a toothbrush, a hairbrush, or a non-round hairbrush.

Exemplary claim 39. The method of any of exemplary claims 31 to 38, wherein the captured digital image frames arc at least one of: one or more digital still images, or one or more digital image frames of a digital video stream.

Exemplary claim 40. The method of any of exemplary claims 31 to 39, wherein determined the shutter trigger sequence controls the camera such that at least some of the captured digital image frames each represent at least one of: a respectively different focused depth profile of the product, or a respectively different focused region of the product.

1. A method of controlling a capture of one or more digital image frames of a product on a conveyor line, the method performed by a manufacturing control device, the manufacturing control device being in communication with at least one camera, the method comprising:
receiving, by the manufacturing control device, a product position trigger signal indicating that the product has entered an imaging zone of the conveyor line;
determining, by the manufacturing control device, a shutter trigger sequence for the at least one camera such that at least a predetermined number of the digital image frames are captured while the product is within the imaging zone of the conveyor line; and
controlling, by the manufacturing control device, the capture of at least the predetermined number of the digital image frames via the at least one camera using the determined shutter trigger sequence upon receipt of the product position signal.

2. The method of claim 1, wherein the determining the shutter trigger sequence for the at least one camera further comprises:
determining, by the manufacturing control device, an image frame duration for each of the predetermined number of the digital image frames.

3. The method of claim 1, wherein the determining the shutter trigger sequence for the at least one camera is based, at least in part, on at least one image frame rate of the at least one camera.

4. The method of claim 1, wherein the at least one camera comprises a fixed focus lens.

5. The method of claim 4, wherein the product is disposed on the conveyor line in at least one of: a vertical orientation, a horizontal orientation, or an intermediate orientation, the method further comprising:
receiving, by the manufacturing control device, a conveyor line speed, wherein the determining the shutter trigger sequence for the at least one camera is based, at least in part, on the conveyor line speed.

6. The method of claim 4, wherein the at least one camera further comprises a liquid lens, the liquid lens having a focus rise time, the method further comprising:
determining, by the manufacturing control device, a focus trigger sequence for the at least one camera such that the at least predetermined number of the digital image frames are captured at one or more focus points while the product is within the imaging zone of the conveyor line, wherein the determining the shutter trigger sequence for the at least one camera is based, at least in part, on the focus rise time.

7. The method of claim 1, wherein the product is disposed on the conveyor line in at least one of: a vertical orientation, a horizontal orientation, or an intermediate orientation, and the conveyor line is stopped during the capture of the at least predetermined number of the digital image frames via the at least one camera.

8. The method of claim 1, wherein the product is at least one of: a toothbrush, a hairbrush, or a non-round hairbrush.

9. The method of claim 1, wherein the captured digital image frames are at least one of: one or more digital still images, or one or more digital image frames of a digital video stream.

10. The method of claim 1, wherein the determined the shutter trigger sequence controls the at least one camera such that at least some of the captured digital image frames each represent at least one of: a respectively different focused depth profile of the product, or a respectively different focused region of the product.

11. A manufacturing control device configured to control a capture of one or more digital image frames of a product on a conveyor line, the manufacturing control device being in communication with at least one camera, the manufacturing control device comprising:
a memory; and
a processor, the processor configured at least to:
receive a product position trigger signal indicating that the product has entered an imaging zone of the conveyor line;
determine a shutter trigger sequence for the at least one camera such that at least a predetermined number of the digital image frames are captured while the product is within the imaging zone of the conveyor line; and
control the capture of at least the predetermined number of the digital image frames via the at least one camera using the determined shutter trigger sequence upon receipt of the product position signal.

12. The device of claim 11, wherein the processor is further configured to:
determine an image frame duration for each of the predetermined number of the digital image frames; and
use the determined image frame duration for the determination of the shutter trigger sequence for the at least one camera.

13. The device of claim 11, wherein the processor is further configured such that the determination of the shutter trigger sequence for the at least one camera is based, at least in part, on at least one image frame rate of the at least one camera.

14. The device of claim 11, wherein the at least one camera comprises a fixed focus lens.

15. The device of claim 14, wherein the product is disposed on the conveyor line in at least one of: a vertical orientation, a horizontal orientation, or an intermediate orientation, the processor being further configured to:
receive a conveyor line speed, wherein the processor is further configured such that the determination of the shutter trigger sequence for the at least one camera is based, at least in part, on the conveyor line speed.

16. The device of claim 14, wherein the at least one camera further comprises a liquid lens, the liquid lens having a focus rise time, the processor being further configured to:
determine a focus trigger sequence for the at least one camera such that the at least predetermined number of the digital image frames are captured at one or more focus points while the product is within the imaging zone of the conveyor line, wherein the processor is further configured such that the determination of the shutter trigger sequence for the at least one camera is based, at least in part, on the focus rise time.

17. The device of claim 11, wherein the product is disposed on the conveyor line in at least one of: a vertical orientation, a horizontal orientation, or an intermediate orientation, and the conveyor line is stopped during the capture of the at least predetermined number of the digital image frames via the at least one camera.

18. The device of claim 11, wherein the processor is further configured such that the determined shutter trigger sequence controls the at least one camera such that at least some of the captured digital image frames each represent at least one of: a respectively different focused depth profile of the product, or a respectively different focused region of the product.

19. A manufacturing control system configured to control a capture of one or more digital image frames of a product on a conveyor line, the system comprising:
a camera;
a position detector; and
a control device, comprising:
a memory; and
a processor, the processor configured at least to:
receive a product position trigger signal from the position detector indicating that the product has entered an imaging zone of the conveyor line;
determine a shutter trigger sequence for the camera such that at least a predetermined number of the digital image frames are captured while the product is within the imaging zone of the conveyor line; and
control the capture of at least the predetermined number of the digital image frames via the camera using the determined shutter trigger sequence upon receipt of the product position signal.

20. (canceled)
